(19) United States
(12) Patent Application Publication
(10) Pub. No.: US 2026/0295166 A1
(43) Pub. Date: Oct. 1, 2026

(54) DRUG DELIVERY DEVICE AND METHOD FOR DECOUPLING A DRUG ADMINISTRATION ELEMENT AND AT LEAST ONE PART OF A DRUG DELIVERY DEVICE

(71) Applicant: SANOFI, Paris (FR)

(72) Inventors: Tim GLÄSSER, Rüsselsheim (DE); Matthias RAU, Rüsselsheim (DE); Michael SCHABBACH, Frankfurt am Main (DE); Nikolai URBAN, Rüsselsheim (DE)

(21) Appl. No.: 19/489,884

(22) PCT Filed: Jun. 5, 2024
(86) PCT No.: PCT/EP2024/065447

§ 371 Date: Dec. 3, 2025

(30) Foreign Application Priority Data
Jun. 5, 2023 (EP) 23315226.3

(51) Int. Cl. A61M 5/24

(52) U.S. Cl. CPC A61M 5/24; A61M 2005/2477; A61M 2205/0216; A61M 2205/21

(57) ABSTRACT

Disclosed is a drug delivery device comprising a housing for receiving a drug reservoir and comprising at least one supporting member. A drug administration element may be fluidically coupled to the drug reservoir. The drug administration element may define a drug outlet of the drug delivery device. The at least one supporting member may be coupled to the drug administration element in order to support the drug administration element relative to the housing. The at least one supporting member may be configured to allow a radial displacement of the housing relative to the drug administration element during a drug delivery operation.

### CROSS-REFERENCE TO RELATED APPLICATIONS

The present application is the national stage entry of International Patent Application No. PCT/EP2024/065447, filed on Jun. 5, 2024, and claims priority to European Application No. EP 23315226.3, filed on Jun. 5, 2023, the disclosures of which are incorporated herein by reference.

### TECHNICAL FIELD

The disclosure relates to a drug delivery device. The drug delivery device may be an autoinjector or a manually or semi-automatically operated device. An energy-storing element may be used in autoinjectors as well as in semi-automatically operated devices in order to deliver the driving force for the injection operation. The energy-storing element may be biased in the factory or by the user prior to use.

The drug may comprise insulin or GLP-1 (Glucagon-Like Peptide). However, other drugs may also be injected.

### BACKGROUND

Self-administration is a great advantage of these devices as it is not necessary to involve medical staff. However, some patients may have reduced dexterity, e.g. due to trembling or reduced flexibility of their fingers or they may be in a hurry, inattentive, etc. Thus, insertion of a needle and/or injection as well as removal of the needle may cause unnecessary pain.

The same issues may be relevant for other insertion and/or injection devices, e.g. lancets, syringes, prefilled syringes, etc.

### SUMMARY

It is an object of the disclosure to provide an improved drug delivery device. The device should be preferably easily and/or comfortably to use and/or comprise as few parts as possible. Furthermore, preferably causes of pain should be reduced, e.g. as much as possible. Furthermore, a corresponding method shall be provided. This object is solved by the drug delivery device disclosed herein.

According to an embodiment, a drug delivery device includes a housing for receiving and/or for retaining a drug reservoir, and at-least one supporting member.

According to a further embodiment, the drug delivery device may be an autoinjector, especially an FLAI (fast and/or large auto-injector). Large may mean that the injection amount may be in the range of 2 ml (milliliter) to 10 ml or in the range of 2 ml to 5 ml or in the range of 2 ml to 3 ml. Fast may mean that even a large amount of medicament is injected within a range of 5 s (second) to 20 s, e.g. less than 20 s for 3 ml or for a volume in the range of 2 ml to 3 ml or e.g. less than 30 s for 5 ml or for a volume in the range of 3 ml to 5 ml.

The viscosity of the medicament may be in the range of 1 cp to 50 cp (centipoise), e.g. in the range of 3 cp to 50cp or in the range of 15 cp to 30 cp. The drug administration element, e.g. a needle may have a diameter ranging from about 25 Gauge to about 31 Gauge in size. Common sizes are 27 and 29 Gauge. However, sizes of 25 Gauge may be uses for fast and large autoinjectors. Larger volumes of medicaments may be used e.g. for medicaments against auto-immune diseases, especially medicaments comprising antibodies, e.g. monoclonal antibodies.

According to an embodiment, the housing may comprise an outer surface that is not surrounded by further parts of the drug delivery device. The outer surface may be a gripping surface that is gripped by a user or patient during usage of the drug delivery device, e.g. during injection of a drug into a body of a patient or of an animal.

According to an embodiment the drug delivery device may have a longitudinal axis. The housing may extend along the longitudinal axis. The housing may comprise a proximal end and a distal end. The distal end may be closer to a needle of the drug delivery device than the proximal end. The housing may comprise only one part or several parts, e.g. a main part, e.g. an elongated cylindrical body and preferably a rear housing part that closes the main part of the housing from its rear side, e.g. at the proximal end of the housing.

According to an embodiment, a drug administration element may be fluidically coupled or may be fluidically coupleable to the drug reservoir. The drug administration element may define or may form a drug outlet of the drug delivery device. A typical example of a drug administration element is a needle. The drug reservoir may be a container, e.g. a cartridge with a distal septum that may be penetrated by a needle, a syringe, a plastic bag or other fluid storage part.

According to an embodiment, the at least one supporting member may be mechanically coupled or may be coupleable to the drug administration element in order to support the drug administration element relative to the housing. There may be an indirect mechanical coupling of the supporting member and of the drug administration element, e.g. only via the drug reservoir or via the drug reservoir and at least one further part of the drug delivery device. Alternatively, there may be direct coupling of the supporting member and of the drug administration element.

According to an embodiment, the at least one supporting member may be configured to allow a radial displacement, e.g. a radial displacement of the housing relative to the drug administration element during a drug delivery operation. Thus, it may be possible to decouple the drug administration element, e.g. needle, from the housing or other part of the drug delivery device or the drug administration element may be decoupled mechanically from the housing or from another part of the drug delivery device. The decoupling may be a decoupling in the radial direction that prevents that radial movements of the housing are transferred to the drug reservoir and/or to the drug administration element, e.g. a needle, preferably during insertion of the needle or other drug administration element and/or during injection of the drug comprised within the drug reservoir and/or during retraction of the needle or other drug administration element and/or during other operations of the drug delivery device. Vice versa, movements, e.g. radial movements of the needle or other drug administration element may not be transferred to the housing or to other part of the drug delivery device, e.g. to the drug reservoir. This decoupling may reduce pain during injection and/or may allow precise injection, e.g. with regard to injection depth and/or injection angle.

The relative radial displacement and/or movement of the drug administration element and/or of the drug reservoir, e.g. of a longitudinal axis of the drug administration element and/or of the drug reservoir may be parallel to a longitudinal axis of the drug delivery device and therewith parallel relative to the housing.

Alternatively, the decoupling may allow pivoting of the longitudinal axis of the drug administration element and/or drug reservoir relative to the longitudinal axis of the housing and/or of other parts of the drug delivery device. A pivoting angle between both axes may be in the range of 1 to 5 degrees or in the range of 1 to 3 degrees or may be at least one degree, at least two degrees or at least three degrees. Angles larger than 3 degrees may be reached with changes of the geometry of the relevant parts. The angle may be less than 20 degrees or less than 10 degrees or less than 6 degrees. The at least one supporting member may include a radial supporting member, e.g. a soft material, radial holding portion (arms) and/or hinged arms, e.g. radially extending arms or radially and axially, e.g. proximally extending arms, and/or an axial supporting member, e.g. supporting arms that extend parallel to or along a plunger, and/or other types of supporting members.

The radial supporting member may support the drug reservoir and/or the drug administration element radially relative to the housing. The axial supporting member may support the drug reservoir and/or the drug administration element axially relative to the housing. Other types of supporting members may support the drug reservoir and/or the drug administration element radially and axially. Details of embodiments of supporting members are mentioned below.

The radial supporting member may provide a retaining space for the at least one drug reservoir within the housing. The radial supporting member may comprise at least one of at least one soft material portion for holding the drug reservoir and/or a at least one flexible holding portion for holding the drug reservoir. The at least one of the at least one soft material and/or of the at least one flexible holding portion may allow a radial displacement of at least a portion of the drug reservoir relative to the housing or of the whole drug reservoir relative to the housing during usage of the drug delivery device. As the drug reservoir may be mechanically coupled to the drug administration element, e.g. in a rigid manner, e.g. needle, the drug administration element may also be decoupled from the housing, e.g. radially.

The relative radial displacement of the drug administration element and/or drug reservoir, e.g. needle, relative to the housing may be at least 0.5 mm (millimeter), at least 1 mm, at least 1.5 mm, at least 2 mm or at least 3 mm and/or may be alternatively or additionally be less than 10 mm or less than 5 mm. Radial displacements of more than 3 mm may be possible with changes of the geometry of the relevant parts.

There may be a relative pivoting movement between the drug administration element/drug reservoir and the housing, e.g. within a range of 1 degree to 5 degree or within a range of 1 degree to 3 degree as mentioned above. Pivoting may take place around a holding point/region in the proximal half or within the proximal third or proximal quarter of the medicament reservoir. Exemplarily, a syringe may have a length with needle of 7.5 mm. The pivoting point (fulcrum) may be at a distance of 50 mm from the outlet of the needle, e.g. the radius. Thus, the circumference of a circle is Pi (3,14) multiplied by the value of the radius of e.g. 50 mm resulting in e.g. 157 mm. A pivoting of 1 degree corresponds to a displacement of about 0.44 mm, a pivoting of 2 degrees corresponds to a displacement of about 0.88 mm and a pivoting of about 3 degrees corresponds to a displacement of about 1.3 mm, etc. in the given example.

The drug reservoir may not be present within the retaining space and/or in the drug delivery device before usage, e.g. during transport of the drug delivery device. Thus, the user has to insert or to assemble the drug reservoir into or onto the drug delivery device, e.g. shortly before injection, e.g. less than one hour before injection of the drug into the body of a patient.

Alternatively, the drug reservoir may be assembled into the drug delivery device during production in a factory. Thus, usage of the drug delivery device may be simple.

According to an embodiment, the at least one supporting member may comprise a radial supporting member that is configured to support the drug reservoir radially during injection of a drug that is comprised in the drug reservoir.

According to a further embodiment, the radial supporting member may support the drug reservoir only at a portion of the drug reservoir that has an axial length that is at most three quarters, at most two thirds, at most one half or at most one third of an axial length of the drug reservoir, preferably of the length of a cylindrical main barrel portion of the drug reservoir or of the overall length of a glass body of the drug reservoir.

According to a further embodiment, the radial supporting member may be configured to support or may support the drug reservoir at a proximal portion of the drug reservoir.

According to an embodiment, the radial supporting member may support the drug administration element and/or the drug reservoir permanently, e.g. during storage of the drug delivery device and/or during injection and at least during injection and/or during retraction.

The radial supporting member may comprise a soft material or a soft hardness material. The soft material may be comprised within at least one portion of soft material. The soft material may have a Shore-A hardness below 90, 85, 80, 75, 70, 65, 60, 55 or below 50. The Shore-A hardness may be more than 5 or more than 10 or more than 15 or more than 20 or more than 30. The Shore-A hardness may be below 90 (e.g. silicone, rubber) or below 85 (e.g. silicone, rubber) or below 80 (e.g. silicone, rubber) or below 75 (e.g. silicone, rubber) or below 70 (e.g. silicone) or below 65 (e.g. silicone) or below 60 (e.g. silicone) or below 55 (silicone) or below 50 (e.g. silicone) or below 40 or below 35. Other materials may be used as well, e.g. foam, rubber, etc. It may be also possible to use a gel, e.g. a gel that is comprised in a plastic bag or in another appropriated flexible bag. The gel may have a Shore hardness 00 in the range of e.g. 45 to 98.

The Shore-hardness measurement method was developed in the year 1915 by Albert Shore. The Shore hardness is a characteristics of material for elastomers and plastics and is defined e.g. within DIN (German Industry Norm/Standard) EN (European Norm/Standard) ISO (International Standardization Organization) 868, DIN ISO 7619-1 and ASTM (originally American Society for Testing and Materials, now an International Organization) D2240-00.

Shore-A harness measurements may be used for soft elastomers, e.g. a measurement using a needle comprising a blunt end. A Shore-durometer may be used to measure Shore-A hardness. A front surface of a truncated cone may have a diameter of 0.79 mm (millimeters). The opening angle of the truncated cone may be 35 degrees. A weight of 1 kg (kilogram) may be used to press the needle into the soft material. The holding time may be 15 seconds. A penetration depth of the needle used for measurement into the material of 2.54 mm (0.1 Inch) or more may correspond to a Shore-A hardness of zero. If the penetration depth is 0 mm, the hardness may correspond to 100 Shore-A. Several scales are defined and may be used for measurement if appropriate.

Shore-D hardness may be used for elastomers as well, e.g. for stiff or tough elastomers. The needle used for measurement may have a ball shaped or a semi-sphere shaped end of a diameter of 0.2 millimeter. The needle may narrow with an angle of 30 degrees. A weight of 5 kg may be used to press the needle into the material. The holding time may be again 15 seconds.

Thus, a Shore-A value of 30 may correspond to a Shore-D value of 6, a Shore-A value of 55 may correspond to a Shore-D value of 14 and a Shore-A value of 75 may correspond to a Shore-D value of 25, etc.

Other methods for determining the hardness may be used as well, e.g. International Rubber HardnessDegree, e.g. named as micro-hardness in Germany.

The soft material may extend form an inner wall of the housing to the retaining space or to the drug reservoir if assembled within the retaining space. Alternatively, an inner housing may be used that may be arranged between an inner surface of the housing and the retaining space and/or the drug reservoir if assembled within the retaining space. The soft material and/or the inner housing may extend in the angular direction along the complete circumference or at least along half of the circumference. There may be several segments of the soft material, e.g. having equal angular distances between two adjacent segments.

Only one portion of soft material, e.g. only one tubular shape portion may be used at an appropriate axial position of the drug delivery device. Alternatively, two or more than two portions of soft material may be used that may be arranged with an axial distance greater 0 mm or greater than 1 mm between adjacent portions along the longitudinal axis of the drug delivery device. The distance of the portions of the soft material may be in the range of 5 mm to 30 mm or in the range of 10 mm to 50 mm or may have another appropriate value. Thus, it may be possible to use less soft material.

A soft material is especially suitable to decouple the drug administration element form the housing. Further, the soft material may also provide some axial support, e.g. if friction between the drug reservoir and the soft material has a large value or an appropriate value.

According to an embodiment, the soft material or the at least one portion of soft material may comprise or may have a tubular shape. An inner lumen portion of the tube may be surrounded by a wall of the tube. The inner lumen portion may extend from a proximal end of the tube to a distal end of the tube. The inner lumen portion of the tube may form a retaining space for retaining the drug reservoir. Thus, the soft material has a simple shape and may be produced easily.

According to an alternative embodiment, the radial supporting member may comprise at least one flexible holding portion. The flexible holding portion may comprise at least one flexible element. The amount of flexibility of the flexible element may be chosen such that bending of the at least one flexible element is possible by radial forces during usage of the drug delivery device. The flexible element may be or may comprise an elongated arm, a beam or a sheet material, etc.

Only one flexible holding portion may be used at an appropriate axial position on the longitudinal axis of the drug delivery device. Alternatively, two or more than two flexible holding portions may be used that may be arranged with an axial distance greater 0 mm or greater than 1 mm between adjacent flexible holding portion along the longitudinal axis of the drug delivery device. The distance may be in the range of 5 mm to 30 mm or in the range of 10 mm to 50 mm or may have another appropriate value.

According to an embodiment, the flexible holding portion may comprise at least one straight element extending radially inwards up to a retaining space for the drug reservoir. Thus, straight arms may be used that may be easily produced, e.g. by using injection molding. Elongated arms may be used, e.g. having circular or elliptical cross section, preferably straight arms.

Alternatively, the flexible holding portion may comprise a spirally wound element. An axis around which a spiral of the spirally wound portion is wound may be parallel to the longitudinal axis drug delivery device or may corresponds to the longitudinal axis of the drug delivery device. The spirally holding portion may be formed as a wounded arm or as a wounded sheet material. The spirally wounded arm may have a circular or elliptical cross section.

According to an embodiment, the at least one supporting member may comprise at least one pre-injection supporting member. The at least one pre-injection supporting member may be configured to be in a first state before the drug administration element is inserted into the tissue of a patient, e.g. into the skin. The at least one pre-injection supporting member may be configured to be in a second state when the drug administration element is inserted into the tissue of a patient. In the first state, the pre-injection supporting member may support the drug administration element and/or the drug reservoir, e.g. radially, only radially or radially and/or axially. In the second state, the pre-injection supporting member may not support the drug administration element or may support the drug administration element to a lesser degree compared to the support in the first state, e.g. the radial supporting force may be reduced by at least 50 percent compared to the holding force in the first state. The pre-injection supporting member may be moved back to the first state or may be allowed to move back to the first state after injection. Alternatively, the pre-injection supporting member may remain in the second state after injection.

The pre-injection supporting member may comprise flexible arms, electronically movable arms, hinged arms, telescopic arms, etc.

During the injection phase an axially movable needle cover may disconnect the pre-injection or stabilizing arms from a drug reservoir and/or from the drug administration element, e.g. from a syringe, thereby decoupling the syringe or other element from radial movement of the body or housing or vice versa thereby decoupling the housing from radial movement of the syringe or of another drug container.

The pre-injection supporting member may be used alone or in combination with other radial supporting members, e.g. the soft material and/or the flexible holding portion.

According to a further embodiment, the drug delivery device may comprise at least one needle cover that is axially movable along the longitudinal axis form a first position to a second position that is proximal with regard to the first position. In the first position, the needle cover may be configured to cover or covers a needle arranged on or arrangeable on the drug reservoir. In its second position, the needle may be configured to expose or may expose the needle. The housing may comprise at least one stabilizing arm that forms the pre-injection supporting member that is configured to stabilize the radial and/or the axial position of the drug reservoir before insertion of the needle arranged on or arrangeable on the drug reservoir into a skin and/or tissue of a patient. The at least one stabilizing arm may extend from the housing or from another part of the drug delivery device into the direction of the drug reservoir and up to the drug reservoir. The at least one stabilizing arm may be arranged in the way of the needle cover during movement of the needle cover from the first position to the second position. Thus, the at least one stabilizing arm may be removed by the needle cover during movement of the needle cover from the first position to the second position. The needle cover may comprise at least one arm, e.g. a long arm that extends along at least half of the axial length of the needle cover and that extends proximally from a distal part of the needle cover. At least two long arms may be used on the needle cover. The at least one long arm of the needle cover may be used to trigger the ejection of the drug or for other purposes.

The needle cover may comprise at least one coupling arm that couples to the pre-injection supporting member, e.g. to the stabilizing arm(s) in order to switch it from the first state to the second state. The coupling arms on the needle cover may also extend proximally form a distal portion of the needle cover, e.g. a needle protection portion. The coupling arms of the needle cover may be much shorter than the at least one long arm extending proximally, e.g. in order to activate drug/medicament injection. Thus, the at least one coupling arm may form a short arm on the needle cover.

Pre-injection support or stabilization may be performed preferably on a distal part or distal portion of the drug reservoir and/or of the drug administration element.

According to an embodiment, the at least one supporting member may comprise an axial supporting member that is configured to support the drug administration element axially, e.g. via the drug reservoir. The axial supporting member may comprise at least one supporting arm that may extend in a distal direction, preferably from a proximal part of the housing, e.g. from a rear part of the housing that was inserted into the housing during assembling of the drug delivery device.

The axial supporting member may extend parallel or essentially parallel to a longitudinal axis of the drug delivery device. “Essentially parallel” may mean that an angle between a longitudinal axis of the axial supporting member and of the longitudinal axis of the drug delivery device is within a range of 0 degrees to 3 degrees, for example. However, other arrangements of the axial supporting member that are not parallel or essentially parallel may also be used.

The drug administration element may be supported axially by the axial supporting member e.g. indirectly via the drug reservoir. Further, the axial supporting member may also provide radial support or it may provide only axial support but no radial support. Both variants are described in more detail below.

The axial support of the axial supporting member may be in both axial directions, e.g. in the distal direction as well as in the proximal direction. Alternatively, the axial support may be in only one axial direction, e.g. only the distal direction but not the proximal direction or only the proximal direction but not the distal direction. The first variant is described below in more detail, e.g. using at least one thread (rope) or filament. The second variant may be feasible in a similar way compared to the first variant.

According to a further embodiment, the drug delivery device may comprise a plunger or piston rod that is configured to transfer a force to a drug comprised within the drug reservoir. The at least one axial supporting member or axial supporting arm may extend along and/or parallel to the plunger/piston rod when the plunger is in a pre-injection state. The at least one axial supporting member or axial supporting arm may have a respective distal portion that is configured to position or that positions the drug reservoir in an axial position. The at least one axial supporting member or axial supporting arm may allow radial movement of a portion of the drug reservoir that is closest to the at least one axial supporting member or to the axial supporting arm or to the at least two axial supporting arms. A relative radial movement of the drug reservoir and/or of the drug administration member relative to the housing and/or relative to the at least one supporting member/arm may be possible.

The axial supporting member may be configured to define/limit the axial position of the drug reservoir and/or of the drug administration member within or relative to the housing or may define/limit axial position of drug reservoir within housing at least into the distal direction or in both the distal direction and the proximal direction.

The axial supporting member/arm may be rigid or flexible, e.g. resiliently flexible.

The axial supporting member/arm(s) may extend from a proximal part of the housing or from a rear part of the housing that is inserted into the housing, as mentioned above. However, alternatively, other parts may be arranged between a proximal portion of the axial supporting member/arm(s) and a proximal part of the housing or between the rear part (rear sub assembly) of housing. This will be described below in more detail, e.g. with regard to flexible portions of coupling elements that may have resiliency, e.g. resilient wires, or may not have resiliency, e.g. filaments.

According to an embodiment, the at least one axial supporting member/arm may carry a low friction material that may enable relative radial movement of the drug reservoir and/or drug administration element relative to the at least one supporting member/arm with lower friction compared to a main material of the at least one supporting arm. The low friction material may comprise or consists of PTFE (Polytetrafluoroethylene) or another appropriate material, e.g. grease, etc. The low friction material may be a separate material layer that may be applied to a basic material of the axial supporting member/arm. The basic material may be different from the low friction material, e.g. two component injection molding may be used to apply the low friction material. The low friction between a proximal end of the drug reservoir, e.g. of a proximal facing face of a flange of the drug reservoir and a distal end of the at least one supporting arm may allow easy radial movement of the drug reservoir and/or of the drug administration element relative to the housing, thereby e.g. reducing pain during insertion of a needle, injection and/or retraction of the needle.

According to an embodiment, the at least one supporting arm may carry a ball bearing or other bearing comprising rolling elements on its distal end, e.g. a proximal ball bearing that may be arranged on a proximal directing surface of a flange of a syringe. The ball bearing may support the drug administration element axially, preferably via the drug reservoir. Further, the ball bearing may allow radial displacement/movement of the drug reservoir/drug administration element relative to the axial supporting member/arm. A flange of a syringe or a proximal face of the drug reservoir may form an abutment face for the balls of the ball bearing. Alternatively, the ball bearing may be mounted on the proximal face of the drug reservoir and the balls may be abut on distal ends of the axial support member/arm(s).

A second or distal ball bearing may be arranged on a distal facing face of the flange of a syringe, thereby providing an improved radial movement degree of freedom. The balls of the ball bearing may abut against a proximal facing surface of the housing. Alternatively, the distal ball bearing may be mounted on the proximal facing surface of the housing or of the inner housing and the balls may abut against a distally facing face of the flange of the syringe.

According to a further embodiment, the drug delivery device may comprise an inner housing that is arranged within the housing that may be named as an outer housing. The inner housing may ease assembling of the drug delivery device or may have other technical effects.

The soft material and/or the flexible holding portion may be arranged at least partially or completely within the inner housing. The soft material and/or the flexible holding portion may be hold by the inner housing.

The inner housing may comprise a proximal directed face that may carry a ball bearing or that may be used as an abutment face for balls of a ball bearing arranged on the flange of a drug reservoir, preferably of a syringe. This ball bearing may be a distal ball bearing that is used in addition to the proximal ball bearing mentioned above. Alternatively, a circumferential flange provided on the inside of the outer housing may be used to carry the ball bearing or to form the abutment face for the balls of the ball bearing, e.g. of the distal ball bearing.

According to an embodiment, the at least one supporting arm may comprise an aperture on its distal end. The aperture may be configured to couple to a flange of the drug reservoir, preferably to the flange of a syringe. The aperture may couple to a part of the flange, e.g. the part of the flange may be placed within the aperture. Backlash (play) between a sidewall of the aperture and the flange may be used to provide degree of freedom for pivoting and/or radial displacement of the drug container/drug administration element. Alternatively, there may be no backlash (play) of the flange portion within the aperture. Nevertheless, relative movement of the drug container relative to the axial supporting member/arm may be possible due to the flexibility of the axial supporting member or due to other reasons.

Again, the supporting arm may extend from a proximal part of the housing or from a rear part of the housing that was inserted into the housing. The supporting arm comprising the aperture may be rigid or flexible. There may be thinner portion of the supporting arm that are thinner compared to adjacent portions of the supporting arm. The thinner portions may promote or increase flexibility of the supporting arm.

According to an embodiment, a proximal end of the at least one supporting arm may be coupled to a proximal part of the housing or to a rear part of the housing, e.g. arear part that was inserted into the housing, by at least one coupling element. The coupling element may be configured to transfer no axial pressure force or less axial pressure force compared to axial tension force transferable by the coupling element. Preferably, the at least one coupling element may comprise or consists of at least one filament. The filament may comprise or may consist of, e.g. plastic, textile or other appropriate material. Several filaments may be woven or braided together. To form the coupling element. Tensile strength of the coupling element may be less than, equal to or more than the tensile strength of supporting arm. Alternatively, a wire may be used, e.g. made of metal or comprising metal. The wire may have resiliency or may not have resiliency. Thus, by using the coupling element it may be possible to provide axial supporting members/arms that may be displaced/moved radially relative to the longitudinal axis of the housing thereby allowing radial displacement of the drug reservoir/drug administration member relative to the housing or vice versa radial displacement of the housing relative to the drug reservoir/drug administration member. Consequently, the axial supporting member may provide its own contribution for reducing pain or providing other medical advantages during usage of the drug delivery device.

The coupling element may bridge an axial distance within the range of 5 mm to 30 mm.

An axial force may be transmitted by the coupling element only or mainly in one direction, e.g. a tension in the distal direction. Other supporting members may provide axial holding forces in the other directions, e.g. as mentioned above: radial supporting member, e.g. soft material, or pre-injection supporting member. The coupling element may be combined with an embodiment of axial supporting arm(s) wherein a distal portion of the supporting arm(s) comprises at least one aperture for the flange of the syringe.

Resiliency may mean that the respective resilient part has a first shape (conformation) before the application of a force. Application of a force may bring the part into a second shape or second conformation. After removal of the force the part may go back or may relax to the first shape (conformation), preferably without application of further forces, i.e. in a self-relaxing manner.

Examples for resiliently flexible materials/parts that may be used as coupling elements are e.g. a plastic sheet spring or a metal sheet spring. Again a tension force may be transmitted. However, a pressure force may only be transmitted by the coupling element up to the point on which sheet spring is deflected, e.g. bended, e.g. resiliently bended.

In summary, a plurality of supporting members is described above, each allowing a reduction of the pain during usage of the drug delivery device or providing other medical advantages and/or technical effects, e.g. with regard to injection depth, injection angle, homogeneity of injection, etc.

According to an embodiment, a retaining space for the drug reservoir and/or the drug reservoir itself may radially be surrounded by a tubular distance space that does not comprise a radial supporting element or that does only comprise at least one pre-injection supporting element that is removed from the drug reservoir before or during injection. The tubular distance space may extend around the drug reservoir or around the retaining space for the drug reservoir and along a length that is at least the axial length of the drug reservoir or that is at least 90 percent of this length. Preferably, the drug reservoir may not be supported radially or is only radially supported at its proximal end during injection of a drug comprised within the drug reservoir.

Thus, support of the drug reservoir and/or drug administration element may be provided only be elements of the drug delivery device that are arranged proximally with regard to the drug reservoir. This may allow free radial “floating” of the drug administration element and/or the drug reservoir e.g. during injection or during other operating phases of the drug delivery device.

The length of the drug reservoir may be measured without the length of a needle or of another drug administration element or including the length of the needle or other drug administration element.

According to a further embodiment, the drug delivery device may comprise a plunger that has a distal end. The distal end of the plunger and a proximal end of a plug within the drug reservoir may be configured to couple with lateral backlash (play), preferably with a lateral backlash (play) of at least 0.5 mm or of at least 1 mm. The plug may comprise a proximal blind hole that is configured to interact with a distal part of the plunger. Alternatively, the proximal surface of the plug may be essentially flat and/or without a special blind hole that interacts with the plug during injection of the drug. The plunger may have a small diameter distal portion that interacts with the blind hole. The diameter of the small diameter portion may be less than the diameter of a main portion of the plunger. The small diameter portion may have a constant diameter and may extend at least 1 mm or at least 2 mm along the longitudinal axis. Alternatively, the plunger may not have a separate small diameter portion.

This embodiment may allow an angle between a longitudinal axis of the plunger/piston rod and an axis of the medicament reservoir, without danger of jamming. However, the plug may be made of a flexible material and may allow angles greater zero degree between both axes without additional backlash (play) or without backlash (play) between a distal portion of the plunger/piston rod and a blind hole within the plug.

According to an embodiment, the housing may be an outer housing of the drug delivery device. According to an embodiment, the drug delivery device may comprise an inner housing that may be arranged within the outer housing. According to an embodiment, the at least one supporting member may be configured to allow also radial displacement of the inner housing relative to the outer housing.

Axial displacement of inner housing relative to outer housing may not be possible or only to a lesser degree compared to radial movement between these two parts. Radial movement may be allowed to a greater degree using this embodiment compared e.g. to other embodiments. According to a further embodiment, a longitudinal axis of a medicament container may be essentially parallel to a longitudinal axis of a plunger/piston rod independent of the radial displacement of the drug administration element relative to the outer housing, especially during injection of the medicament/drug, e.g. during the whole injection phase. This may avoid contact between an inner surface of the medicament container and an outer surface of the plunger/piston rod during injection of the medicament. Thus, injection may be performed in a repeatable and secure/safe manner. Breakage of the medicament container may be prevented, e.g. of a glass part of the medicament container.

The at least one supporting member may comprise a soft material portion and/or flexible holding elements, e.g. arms or beams or a sheet of material, see embodiments mentioned above. Alternatively, a gel may be used.

According to an embodiment, the drug delivery device may comprise an axial support element that is arranged on the outer housing. According to an embodiment, the axial support element may be configured to allow radial displacement of a proximal end of the inner housing relative to a proximal end of the outer housing. Thus, the radial displacement of the needle or drug administration element relative to the housing may be made greater.

According to a further embodiment, the axial support element may comprise a surface having a low friction coefficient, e.g. a surface comprising Teflon (may be a trademark) or coated with a low friction material. In an embodiment the axial support element may comprise a bearing, especially a bearing comprising rotation elements, e.g. balls, cylinders, etc.

According to a further embodiment, the axial support element may comprise at least one resilient element, e.g. at least one spring element, especially a spring element comprising plastic or consisting of plastic. However, other materials may be use as well, e.g. a spring element comprising a metal or consisting of a metal.

According to an embodiment, the drug delivery device includes at least one or both of a drive spring that may be configured to drive a plunger or the plunger distally, or an axially movable needle cover that may be axially movable along a longitudinal axis of the housing or of the drug delivery device form a first position to a second position that is proximal with regard to the first position.

Thus, the drug delivery device may be an auto-injector allowing easy injection, e.g. no manual driving force may be necessary to inject drug.

In the first position, the needle cover may be configured to cover or may cover a needle (or another drug administration element) arranged on or arrangeable on the drug reservoir. In its second position, the needle cover may be configured to expose or exposes the needle. The needle cover may trigger and/or may allow triggering of drug delivery, e.g. by unlocking the plunger from a locked position/state.

According to a further embodiment, a method to decouple a needle or other drug administration element from at least one part of a drug delivery device is disclosed. Preferably, the method may be performed to decouple the drug administration element from at least one part of a drug delivery device according to any one of the preceding embodiments. Thus, the same technical effects mentioned above for the drug delivery device may apply to the method.

The method may comprise:
Using a radial supporting member comprising at least one of at least one soft material for holding the drug reservoir or at least one flexible holding portion for holding the drug reservoir, wherein the at least one of the at least one soft material or the at least one flexible holding portion may be configured to allow a relative radial displacement of at least a portion of the drug reservoir or of the whole drug reservoir relative to a housing of the drug delivery device during insertion of a drug administration element arranged or arrangeable on the distal end of the drug reservoir into the tissue of a patient.

Thus, the same technical effects mentioned above for the drug delivery device may apply to the method.

The making and using of the presently preferred embodiments are discussed in detail below. It should be appreciated, however, that the present disclosure provides many applicable concepts that can be embodied in a wide variety of specific contexts. The specific embodiments discussed are merely illustrative of specific ways to make and use the disclosed concepts, and do not limit the scope of the claims.

Moreover, same reference numerals refer to same technical features if not stated otherwise. As far as “may” is used in this description it means or it may mean the possibility of doing so as well as the actual technical implementation. The present concepts of the present disclosure will be described with respect to preferred embodiments below in a more specific context namely drug delivery devices, especially drug delivery devices for humans or animals. The disclosed concepts may also be applied, however, to other situations and/or arrangements as well, e.g. for lancets, syringes, prefilled syringes or other injectors.

The foregoing has outlined rather broadly the features and technical advantages of embodiments of the present disclosure. Additional features and advantages of embodiments of the present disclosure will be described hereinafter, e.g. of the subject-matter of dependent claims. It should be appreciated by those skilled in the art that the conception and specific embodiments disclosed may be readily utilized as a basis for modifying or designing other structures or processes for realizing concepts which have the same or similar purposes as the concepts specifically discussed herein. It should also be recognized by those skilled in the art that equivalent constructions do not depart from the spirit and scope of the disclosure, such as defined in the appended claims.

### BRIEF DESCRIPTION OF THE DRAWINGS

For a more complete understanding of the presently disclosed concepts and the advantages thereof, reference is now made to the following description in conjunction with the accompanying drawings. The drawings are not drawn to scale. In the drawings the following is shown in:

FIGS. 1A to 1D a cross section of a drug delivery device according to a first embodiment and in different operating states,

FIG. 2 a general second embodiment of a drug delivery device,

FIGS. 3A to 3C a third embodiment of a drug delivery device,

FIG. 4 a fourth embodiment of a drug delivery device,

FIG. 5 a fifth embodiment of a drug delivery device,

FIGS. 6A to 6E variants of further embodiments,

FIG. 7 a last embodiment, and

FIG. 8 an expanded structural formula, molecular formula, and molecular weight of fitusiran.

### DETAILED DESCRIPTION

Reference may be made to a cylindrical coordinate system, i.e. each position may be defined by three coordinates: axial value (height, distance to zero plane), radial distance to axis and angle between current radial position and a plane that is defined as having angle zero. In this document the words “in an axial position” may mean having an axial coordinate.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

The distal direction may be the direction in which a tip of the needle is oriented to. A proximal direction may be a direction opposite the distal direction. The distal and proximal directions may be aligned along a main axis. The main axis may be colinear with the needle or run through the needle. Any or all moving components of the injection device may be configured to move along the main axis and/or rotate around the main axis if not described otherwise. A radial direction may be any direction orthogonal to the main axis. The main axis may be an axis along which most of the components of the injection device are arranged and/or oriented. The main axis may be an axis of lowest moment of inertia of the injection device or at least close to such an axis.

Reference signs having the same last two digits may refer to same or similar elements if not mentioned otherwise, e.g. main housing part 102 and 302.

Embodiments may relate to drug delivery devices comprising other activation mechanism or operated by a manual driving force. Reference is made e.g. to WO 2014/033195 A1 or to WO 2014/033197 A1 incorporated herein by reference. The injection button may provide at least one user interface member for initiating and/or performing a dose delivery operation of the drug delivery device. The (dial) grip or knob may provide a user interface member for initiating and/or performing a dose setting operation using a dose setting surface, e.g. the circumferential surface of the (dial) grip or knob. A delivery surface may be used to initiate dose delivery. The delivery surface may be the proximal P surface of the (dial) grip or knob.

The device may be of the dial extension type, i.e. its length may increase during dose setting or dose dialing. Other injection devices with the same kinematical characteristic of the dial extension and button during dose setting and dose expelling operational mode are known as, for example, Kwikpen® and Savvio® device marketed by Eli Lilly as well as FlexPen®, FlexTouch® and Novopen® 4 device marketed by Novo Nordisk or devices of other manufacturers. An application of the general principles disclosed herein to these devices therefore appears straightforward and further explanations will be omitted. Alternatively, the proposed concepts may be used in devices that are not of the dial extension type but include for instance a torsion spring that may be biased by rotation of a dial knob. Moreover, fully mechanically driven or electromechanically driven drug delivery devices may be used, e.g. comprising an electrical motor.

However, the general principles of the present disclosure are not limited to that kinematical characteristic. Certain other embodiments may be conceived, e.g. for application to other injection devices of Sanofi or devices of other manufacturers where there is a container for a drug that may be moved axially relative to a housing of the drug delivery device in order to insert and/or to retract a needle.

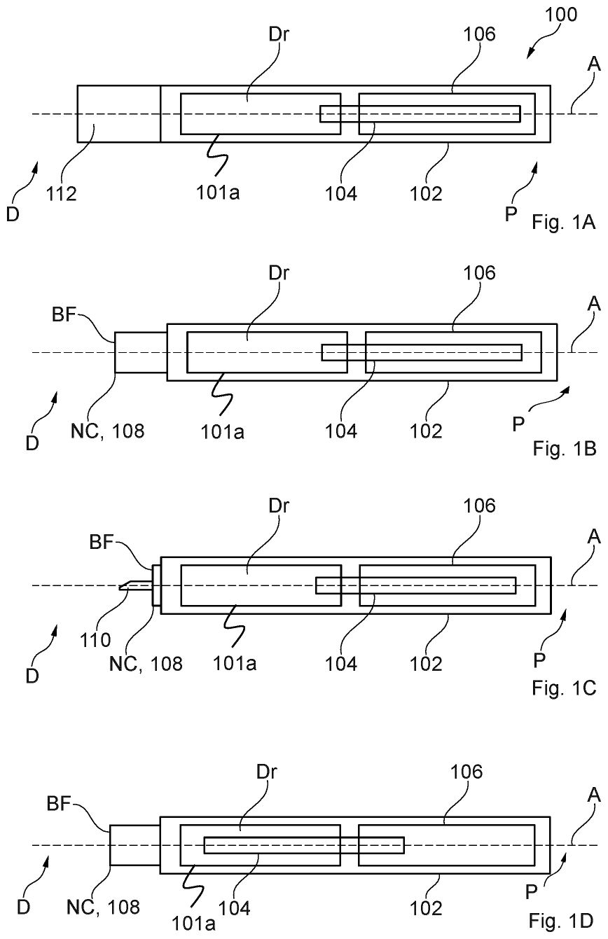

FIGS. 1A through 1D illustrate an embodiment of a drug delivery device 100. Device 100 may be suitable as the device in the drug delivery arrangements described further above and below. The figures show device 100 in different states during its operation.

FIG. 1A illustrates drug delivery device 100 in an initial or as delivered state. Drug delivery device 100 may comprise a housing 102. Housing 102 may be provided to retain and/or may retain a medicament/drug reservoir 101a in its interior. Medicament, e.g. liquid medicament or drug Dr, may be arranged in drug reservoir 101a. Housing 102 may be configured or may be provided to retain and/or may retain a needle 110, see FIG. 1C. The needle 110 may be named as a drug administration element in the claims. However, other drug administrations elements are possible as well, e.g. a nozzle. In other words, needle 110 may be arranged or may be arrangeable in housing 102. Needle 110 may be an integral part of drug reservoir 101a, e.g. (permanently or releasably) connected to a body of drug reservoir 101a, or separate from drug reservoir 101a. In the first case, the drug reservoir 101a may be a syringe, e.g. a PFS (Pre Filled Syringe). In the second case, the drug reservoir 101a may be a cartridge. In case a cartridge is used as drug reservoir 101a, initially, drug reservoir 101a and needle 110 can be fluidly disconnected and fluid communication between the interior of drug reservoir 101a and needle 110 may only be established during operation of drug delivery device 100.

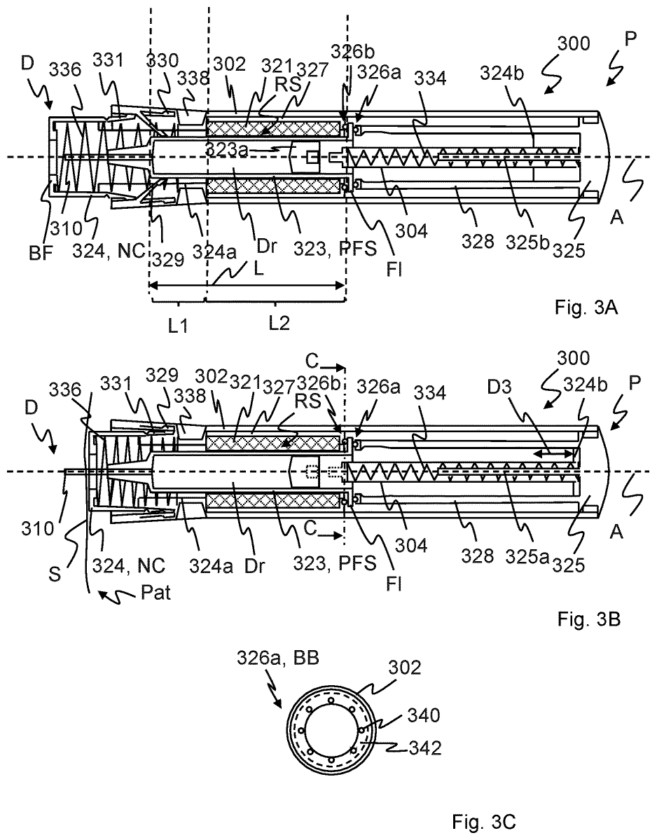

A drive mechanism 106 may be provided to drive a drug delivery operation and may expediently be provided in housing 102. Drive mechanism 106 may comprise a plunger rod 104. Drug delivery device 100 may further comprise a drive energy source, e.g. a drive spring, such as a compression spring, (not explicitly shown) or gas or compressed air. The drive energy source may be arranged to drive plunger rod 104 in a distal direction D relative to drug reservoir 101a during the drug delivery operation. During this movement, a stopper or plug (see e.g. stopper (plug) 323a in FIG. 3), which may be movably retained in drug reservoir 101a and which may seal the medicament container proximally, may be displaced towards an outlet of drug reservoir 101a to dispense drug Dr or medicament retained within the drug reservoir 101a through the outlet. The outlet may be formed or defined by needle 110, see FIG. 1C.

Other potential drive energy sources different from a spring comprise an electrical power cell or battery for driving plunger rod 104 by a motor or a reservoir suitable to provide gas pressure, where the gas pressure can be used to drive the drug delivery operation.

Drug delivery device 100 may be an autoinjector. The energy for driving the drug delivery operation in an autoinjector may be provided by components integral to drug delivery device 100 and does not have to be loaded into the device by the user during the operation of device 100 as is the case in many spring driven pen-type variable dose injectors, where, usually, the energy is loaded into the spring by the user during a dose setting procedure.

Drug delivery device 100 may expediently be a single shot device, i.e. it is provided to dispense only one dose. Drug delivery device 100 may be a disposable drug delivery device 100, that is to say a device 100 which is disposed of after its use. Device 100 may be a pen-type device. A drug reservoir 101a and/or needle 110 may be axially secured within drug delivery device 100, e.g. within housing 102, or may be movable relative to housing 102, e.g. for piercing the skin. In the first case, the user may have to perform the movement for piercing the skin with needle 110. In the second case, piercing of the skin by needle 110 may be driven by a needle insertion mechanism of drug delivery device 100. Automatic needle retraction may be used as well. Drug reservoir 101a may be hold or retained within housing 102 by an optional retaining/supporting member 101b, e.g. by a syringe carrier or other carrier for a drug reservoir 101a. Other embodiments do not comprise an additional syringe carrier or other drug reservoir carrier in addition to the syringe and to housing 102. Thus, drug reservoir 101a may be carried directly by housing 102, e.g. as described below in more detail.

As depicted in FIG. 1A, drug delivery device 100 may further comprises a cap 112. Cap 112 may be arranged at the distal end D of drug delivery device 100. Cap 112 may be detachably connected to the remainder of device 100, e.g. to housing 102 and/or to another component or member of drug delivery device 100. Cap 112 may cover a distal end D of the remainder of drug delivery device 100 and/or a needle passage opening through which needle 110, e.g. the distal needle tip, may pass to pierce the skin from the interior of drug delivery device 100 during or for the drug delivery operation. Cap 112 may comprise a needle cover/shield remover or grabber, e.g. made of metal, which may engage a rigid needle shield/cover RNS, which may cover needle 110 such that the rigid needle shield RNS is removed from needle 100 together with cap 112, e.g. when cap 112 is detached or disconnected from device 100. Housing 102 may expediently cover the majority of the length of drug delivery device 100, e.g. 60 percent or 70 percent or more percent of the entire length of the drug delivery device 100 (with the cap 12 attached and/or with the cap detached).

FIG. 1B illustrates drug delivery device 100 with cap 112 being removed. According to FIG. 1B, device 100 may be in a state ready to be operated, e.g. ready to perform a drug delivery operation when the operation is triggered. As depicted, drug delivery device 100 may further comprises a needle cover NC. Needle cover NC may protrude distally from housing 102 and/or may have been covered by cap 112 when cap 112 was still attached to housing 102. Needle cover NC may be axially movable relative to housing 102 from an initial position or first position to a second position or trigger position. Needle cover NC may be provided to extend beyond the distal tip of needle 110 which may protrude from the housing 102 before the drug delivery operation is commenced, e.g. in a first position. Needle cover NC may be movable in the proximal direction relative to housing 102. During this movement, e.g. before needle cover NC reaches the second position, needle 110 may pierce the skin of the user.

Needle cover NC may serve as a trigger member 108 of the drug delivery device. Needle cover NC as trigger member 108, when displaced proximally from the initial position or first position depicted in FIG. 1B to the second position or trigger position (see FIG. 1C), may automatically initialize the drug delivery operation of drug Dr, preferably when it is in the second position. The drug delivery operation can be initialized by removing a mechanical lock which prevents movement of plunger rod 104 in the distal direction or by moving plunger rod 104 to disengage a mechanical lock using moving needle cover NC. Alternatively, needle cover NC when moved from the first position to the second position and expediently when in the second position may only enable triggering of the drug delivery operation. In this case, a separate trigger member, e.g. a trigger button on the proximal end of the housing 102, may be provided to initiate the drug delivery operation. Operating the trigger button to initiate the drug delivery operation of drug Dr may only be possible when needle cover NC is in the second position. In yet another alternative, needle cover NC may only be provided to prevent needle stick injuries before and/or after use of drug delivery device 100. In this case, needle cover NC may be completely decoupled from drive mechanism 106 and/or may not be involved in triggering or enabling triggering of the drug delivery operation at all.

Needle cover NC may be provided to bear against the skin of a user during injection. Hence, the distal surface of needle cover NC may provide a bearing surface or bearing face BF. Bearing surface BF may delimit and/or extend around a needle passage opening provided in needle cover NC. The bearing surface 16 may be ring-like, oval, elliptic, rectangular, quadratic, etc., circumferentially closed and/or be defined by an inward protrusion protruding radially inwards from an inner wall of needle cover NC, e.g. a distal cylindrical portion thereof. Bearing surface BF may be expediently the distal end surface of needle cover NC, e.g. facing distally.

FIG. 1C illustrates needle cover NC in the second position relative to housing 102. This is the position when the drug delivery operation has been initiated, can be initiated, and/or when the needle pierces the skin, for example. Needle 110 may protrude axially from bearing surface BF of drug delivery device 100 (particularly through the needle passage opening in needle cover NC) and, by the distance with which it protrudes over the bearing surface BF, penetrate the skin (the skin is not shown in this representation, see e.g. skin S in FIG. 3B). This distance may be characteristic for or be equal to the injection depth. The device 100 may be maintained in contact with the skin until the drug delivery operation of drug Dr has been completed, which may be indicated by an optional audible, tactile, and/or visual indication provided by the drug delivery device 100. Optionally, also the beginning of drug delivery may be signaled to the user.

After the drug delivery operation of drug Dr has been completed, e.g. piston rod 104 has moved distally, device 100 may be removed from the skin (see FIG. 1D). Needle cover NC may be biased relative to housing 102 towards the first position by a spring (not shown, see e.g. spring 336 in FIG. 3A). Thus, when device 100 is removed from the skin, needle cover NC may be moved towards the first position with respect to the housing 102. Needle cover NC may be moved distally, e.g. beyond its first position, into a final, third or locked position relative to housing 102. In this position, needle cover NC may expediently be locked axially relative to the housing 102 against movement in the proximal direction P, e.g. by a locking engagement between a locking feature of needle cover NC and housing 102. As it is axially locked, needle cover NC may no longer be displaced proximally relative to housing 102 into the second position and/or into the first position. This may protect the user from needle stick injuries after use. In this state, device 100 may be locked, see FIG. 1D.

Alternatively, there may be no movable needle cover. Thus, a distal device portion may be pressed against the skin of a user. Needle injection may be performed by a needle insertion mechanism. An optional needle retraction mechanism may also be used. Triggering may be performed using a proximal trigger element, e.g. a proximal knob of modified device 100.

Drug delivery device 100 may comprise an electronic unit that may be mechanically connected to a proximal end region P or to another region of drug delivery device 100. The electronic unit may be used not only for drug delivery device 100 but also for other drug delivery devices that are similar or identical to drug delivery device 100. Alternatively, the electronic unit may be an integrated part of the drug delivery device 100. The electronic unit may be used to monitor drug delivery, e.g. amount of dose, time and date.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of pharmaceutical formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug reservoir” adapted for use with a drug delivery device. The drug reservoir 101a may be, e.g., a cartridge, syringe, reservoir, or other solid or flexible vessel (bag) configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug reservoir may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro (B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala (B26) human insulin; Des(B28-B30) human insulin; Des(B27) human insulin and Des(B30) human insulin.

Examples of insulin derivatives are, for example, B29-N-myristoyl-des (B30) human insulin, Lys(B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N-(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F (ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Further examples of APIs for the prophylaxis of hemophilia A or B, with or without inhibitors, include an siRNA targeting antithrombin. An example of an siRNA targeting antithrombin is fitusiran. The term “prophylaxis” and “prophylactic treatment” are used interchangeably herein

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, pharmaceutical formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

Fitusiran as the API for the medicament in the device

Fitusiran is a synthetic, chemically modified double-stranded small interfering RNA (siRNA) oligonucleotide covalently linked to a tri-antennary N-acetyl-galactosamine (GalNAc) ligand targeting AT3 mRNA in the liver, thereby suppressing the synthesis of antithrombin. See, e.g., Pasi et al., N Engl J Med. (2017) 377 (9):819-28. The nucleosides in each strand of fitusiran are connected through either 3′-5′ phosphodiester or phosphorothioate linkages, thus forming the sugar-phosphate backbone of the oligonucleotide.

The sense strand and the antisense strand contain 21 and 23 nucleotides, respectively. The 3′ end of the sense strand is conjugated to the GalNAc containing moiety (referred to herein as L96) through a phosphodiester linkage. The sense strand contains two consecutive phosphorothioate linkages at its 5′ end. The antisense strand contains four phosphorothioate linkages, two at the 3′ end and two at the 5′ end. The 21 nucleotides of the sense strand hybridize with the complementary 21 nucleotides of the antisense strand, thus forming 21 nucleotide base pairs and a two-base overhang at the 3′-end of the antisense strand. See also U.S. Pat. Nos. 9,127,274, 11,091,759, US2020/0163987A1, and WO 2019/014187, the entire contents each of which are expressly incorporated herein by reference.

The two nucleotide strands of fitusiran are shown below:

sense strand:
(SEQ ID NO: 1)
5′Gf-ps-Gm-ps-Uf-Um-Af-Am-Cf-Am-Cf-Cf-Af-Um-Uf-
Um-Af-Cm-Uf-Um-Cf-Am-Af-L96 3′,
and
antisense strand:
(SEQ ID NO: 2)
5′ Um-ps-Uf-ps-Gm-Af-Am-Gf-Um-Af-Am-Af-Um-Gm-Gm-
Uf-Gm-Uf-Um-Af-Am-Cf-Cm-ps-Am-ps-Gm 3′,

wherein
Af=2′-deoxy-2′-fluoroadenosine
Cf=2′-deoxy-2′-fluorocytidine
Gf=2′-deoxy-2′-fluoroguanosine
Uf=2′-deoxy-2′-fluorouridine
Am=2′-O-methyladenosine
Cm=2′-O-methylcytidine
Gm=2′-O-methylguanosine
Um=2′-O-methyluridine
“-” (hyphen)=3′-5′ phosphodiester linkage sodium salt
“-ps-”=3′-5′ phosphorothioate linkage sodium salt
   and wherein L96 has the following formula:

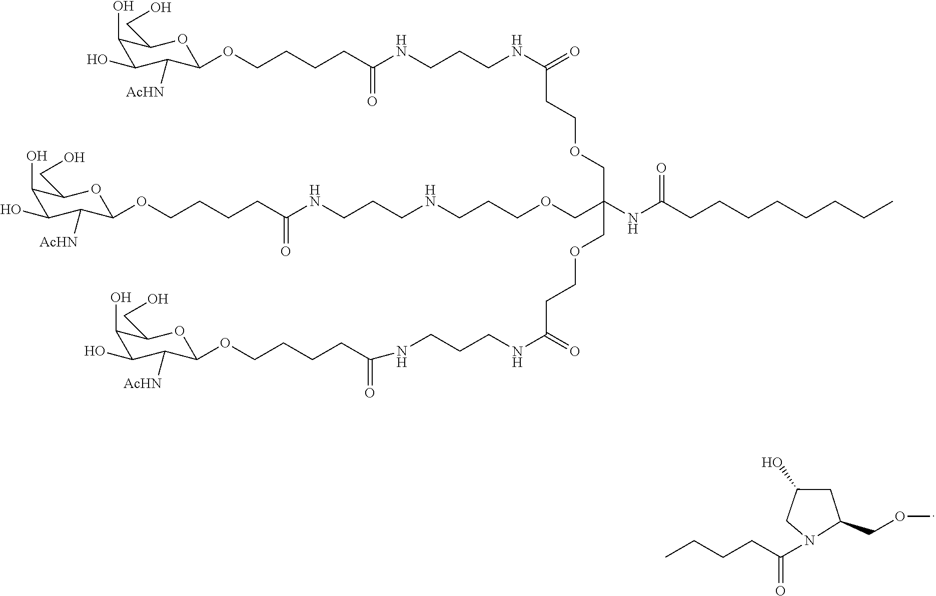

As used herein, the terms 2′-deoxy-2′-fluoroadenosine and 2′-fluoroadenosine may be used interchangeably.

As used herein, the terms 2′-deoxy-2′-fluorocytidine and 2′-fluorocytidine may be used interchangeably.

As used herein, the terms 2′-deoxy-2′-fluoroguanosine and 2′-fluoroguanosine may be used interchangeably.

As used herein, the terms 2′-deoxy-2′-fluorouridine and 2′-fluorouridine may be used interchangeably.

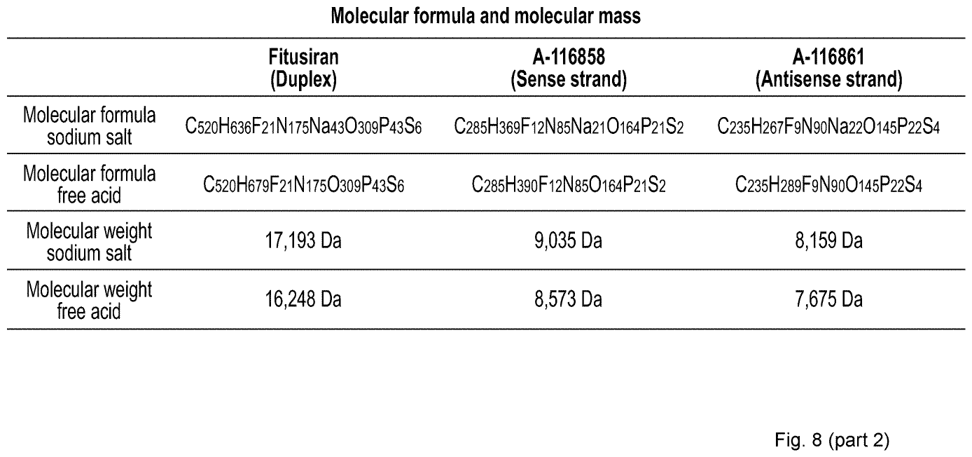

The expanded structural formula, molecular formula, and molecular weight of fitusiran are shown in FIG. 8.

The structure of fitusiran can also be described using the following diagram, wherein the X is O:

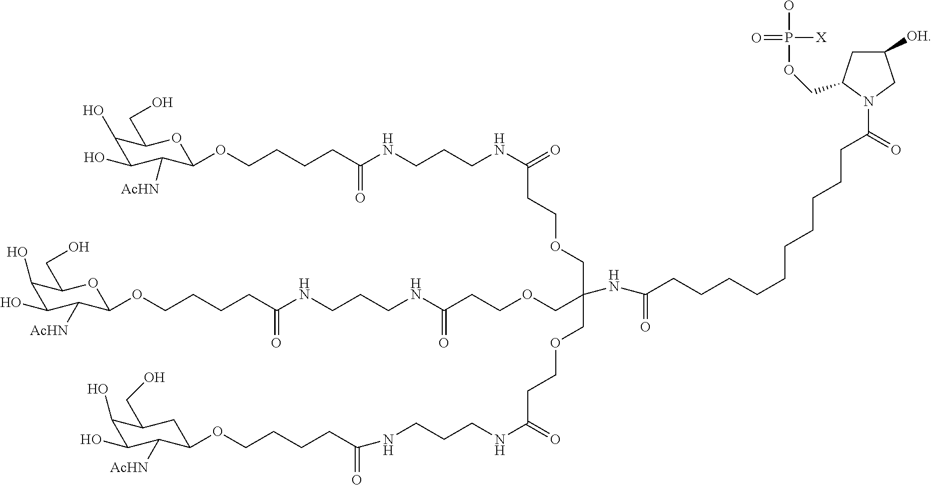

Fitusiran is shown in FIG. 8 in sodium salt form.

In some embodiments, the device delivers fitusiran in an aqueous solution, wherein fitusiran is at a concentration of about 40 to about 200 mg/ml (e.g., about 50 to about 150 mg/ml, about 80 to about 110 mg/ml, or about 90 to about 110 mg/mL). As used herein, values intermediate to recited ranges and values are also intended to be part of this disclosure. In addition, ranges of values using a combination of any of recited values as upper and/or lower limits are intended to be included. In further embodiments, the pharmaceutical formulation comprises fitusiran in an aqueous solution at a concentration of about 40, about 50, about 75, about 100, about 125, about 150, or about 200 mg/ml. In certain embodiments, fitusiran is provided in an aqueous solution at a concentration of about 100 mg/mL.

The term “deliver,” “delivers,” or “delivering” is intended to mean “administer,” “administers,” or “administering.”

Unless specifically stated or otherwise evident from the context, as used herein, the term “approximately” or “about” refers to a value that is within an acceptable error range for a particular value determined by a person of ordinary skill, a portion of which will depend on how the measurement or determination is made. For example, “approximately” or “about” may mean a range of up to 10% (ie, ±10%). Therefore, “approximately” or “about” can be understood as greater than or less than 10%, 9%, 8%, 7%, 6%, 5%, 4%, 3%, 2%, 1%, 0.5%, 0.1%, 0.05%, 0.01%, or 0.001%. When a specific value is provided in this disclosure, unless otherwise stated, the meaning of “approximately” or “about” should be assumed to be within an acceptable error range for that specific value.

While the fitusiran dosage weight described herein refers to the weight of fitusiran free acid (active moiety), administration of fitusiran to patients herein refers to administration of fitusiran sodium (drug substance) provided in a pharmaceutically suitable aqueous solution (e.g., a phosphate-buffered saline at a physiological pH). For example, about 100 mg/ml fitusiran means about 100 mg of fitusiran free acid (equivalent to about 106 mg fitusiran sodium, the drug substance) per mL. Unless otherwise indicated, a fitusiran weight recited in the present disclosure is the weight of fitusiran free acid (the active moiety).

In some embodiments, a pharmaceutical formulation in the device comprises fitusiran in a phosphate-buffered saline. The phosphate concentration in the solution may be about 1 to about 10 mM (e.g., about 2, about 3, about 4, about 5, about 6, about 7, about 8, or about 9 mM), with a pH of about 6.0-8.0. The pharmaceutical formulations herein may include a stabilizing agent such as EDTA. The pharmaceutical formulations may be preservative-free. In some embodiments, the fitusiran pharmaceutical formulation in the device is preservative-free and comprises, consists of, or consists essentially of about 100 mg of fitusiran per ml of an approximately 5 mM phosphate buffered saline (PBS) solution. In some embodiments, the fitusiran pharmaceutical formulation in the device is preservative-free and comprises, consists of, or consists essentially of fitusiran in an approximately 5 mM phosphate buffered saline (PBS) solution. The PBS solution is composed of sodium chloride, dibasic sodium phosphate (heptahydrate), and monobasic sodium phosphate (monohydrate). Sodium hydroxide solution and diluted phosphoric acid may be used to adjust the pH of the pharmaceutical formulation to about 7.0 or about 7.1.

In some embodiments, the fitusiran pharmaceutical formulation in the device for subcutaneous delivery contains fitusiran in a 5 mM phosphate buffered saline having 0.64 mM NaH₂PO₄, 4.36 mM Na₂HPO₄, and 84 mM NaCl at pH 7.0. In certain embodiments, the pharmaceutical formulation of fitusiran solution for subcutaneous delivery is shown in Table 1 below:

TABLE 1
Exemplary Fitusiran Pharmaceutical Formulation

Pharmaceutical Formulation

| | Percentage | Per ml |
|---|---|---|
| Components | [%] | [mg] |
| Fitusiran (active moiety) | 10 | 100 |
| [equivalent to fitusiran sodium] | | [106] |
| Sodium chloride | 0.49 | 4.909 |
| Dibasic sodium phosphate | 0.12 | 1.169 |
| (heptahydrate) | | |
| Monobasic sodium phosphate | <0.01 | 0.0885 |
| (monohydrate) | | |
| Phosphoric acid, concentrated | — | q.s. pH 7.0 |
| Sodium hydroxide | — | q.s. pH 7.0 |
| Water for subcutaneous | q.s. 100 | q.s. 1 mL |
| delivery | | |

*q.s.: quantum satis

In some embodiments, the pharmaceutical formulation of fitusiran solution for subcutaneous delivery with the device can be described as shown in Table 2 below.

TABLE 2
Exemplary Fitusiran Pharmaceutical Formulation

| | Pharmaceutical Formulation |
|---|---|
| Components | (mg) |
| Fitusiran (active moiety) | 100 |
| [equivalent to fitusiran sodium] | [106] |
| NaH₂PO₄*H₂O | 0.0885 |
| Na₂HPO₄*7H₂O | 1.169 |
| NaCl | 4.909 |
| 0.1N NaOH | q.s. |
| 0.1M H₃PO₄ | q.s. |
| Purified water | Ad 1 mL |

In some embodiments, the device may be used to deliver a single dose of fitusiran wherein the single dose comprises about 20 to about 80 mg of fitusiran (e.g., about 20 mg, about 25 mg, about 30 mg, about 40 mg, about 50 mg, or about 80 mg). In some embodiments, the device may be used to deliver single dose of fitusiran, wherein the single dose comprises about 1 to about 30 mg of fitusiran (e.g., about 1.25 mg, about 2.5 mg, about 5 mg, about 10 mg, about 20 mg, or about 30 mg).

In one embodiment, the device may be used to deliver a single dose of about 80 mg of fitusiran. In one embodiment, the device may be used to deliver a single dose of about 50 mg of fitusiran. In one embodiment, the device may be used to deliver a single dose of about 20 mg of fitusiran. In one embodiment, the device may be used to deliver a single dose of about 30 mg of fitusiran. In one embodiment, the device may be used to deliver a single dose of about 10 mg of fitusiran. In one embodiment, the device may be used to deliver a single dose of about 5 mg of fitusiran. In one embodiment, the device may be used to deliver a single dose of about 2.5 mg of fitusiran. In one embodiment, the device may be used to deliver a single dose of about 1.25 mg of fitusiran.

In some embodiments, the single dose of fitusiran may be delivered in about 0.5 mL to about 1 mL delivery volumes (e.g., about 0.5 mL, about 0.6 mL, about 0.7 mL, about 0.8 mL, about 0.9 mL, or about 1 mL). Other delivery volumes described herein may also be used.

In one embodiment, the device may be used to deliver a single dose of about 80 mg of fitusiran in about 0.8 mL (about 100 mg fitusiran/mL). In one embodiment, the device may be used to deliver a single dose of about 50 mg of fitusiran in about 0.5 ml (about 100 mg fitusiran/mL). In one embodiment, the device may be used to deliver a single dose of about 20 mg of fitusiran in about 0.5 ml (about 40 mg fitusiran/mL). In one embodiment, the device may be used to deliver a single dose of about 30 mg of fitusiran in about 0.5 ml (about 60 mg fitusiran/mL). In one embodiment, the device may be used to deliver a single dose of about 10 mg of fitusiran in about 0.5 ml (about 20 mg fitusiran/mL). In one embodiment, the device may be used to deliver a single dose of about 5 mg of fitusiran in about 0.5 mL (about 10 mg fitusiran/mL). In one embodiment, the device may be used to deliver a single dose of about 2.5 mg of fitusiran in about 0.5 ml (about 5 mg fitusiran/mL). In one embodiment, the device may be used to deliver a single dose of about 1.25 mg of fitusiran in about 0.5 ml (about 2.5 mg fitusiran/mL).

In one embodiment, the device delivers fitusiran at a prophylactically effective amount to prophylactically treat hemophilia (e.g., hemophilia A or B, in a patient with or without inhibitors) in a patient in need thereof (e.g., a hemophilia A or B patient, with or without inhibitors). “Prophylactically effective amount” refers to the amount of fitusiran that helps the patient with hemophilia A or B, with or without inhibitors to achieve a desired clinical endpoint such as reducing the Annualized Bleeding Rate (ABR), Annualized Joint Bleeding Rate (AjBR), Annualized Spontaneous Bleeding Rate (AsBR), or the frequency of bleeding episodes. As used herein in the context of fitusiran, the term “treat” “treating,” or “treatment” includes prophylactic treatment of the disease and refers to achievement of a desired clinical endpoint.

A hemophilia A or B patient with inhibitors refers to a patient who has developed alloantibodies to the factor he/she has previously received (e.g., factor VIII for hemophilia A patients or factor IX for hemophilia B patients). A hemophilia A or B patient with inhibitors may become refractory to replacement coagulation factor therapies. A patient without inhibitors refers to a patient who does not have such alloantibodies. The present treatment methods may be beneficial for hemophilia A patients with inhibitors, as well as for hemophilia B patients with inhibitors.

As used herein, a patient with “hemophilia A or B, with or without inhibitors,” or refers to 1) a hemophilia A patient with inhibitors, or 2) a hemophilia B patient with inhibitors, 3) a hemophilia A patient without inhibitors, or 4) a hemophilia B patient without inhibitors. As used herein, a patient refers to a human patient. A patient can also refer to a human subject.

In some embodiments, the device may be used to prophylactically treat a patient with hemophilia A or B, with or without inhibitors, with a subcutaneous dose of about 50 mg of fitusiran once every two months (or every eight weeks). In other embodiments, the device may be used to prophylactically treat a patient with hemophilia A or B, with or without inhibitors, with a subcutaneous dose of about 50 mg of fitusiran every month (or every four weeks). In yet other embodiments, the device may be used to prophylactically treat a patient with hemophilia A or B, with or without inhibitors, with a subcutaneous dose of about 80 mg of fitusiran every two months (or every eight weeks). In yet other embodiments, the device may be used to prophylactically treat a patient with hemophilia A or B, with or without inhibitors, with a subcutaneous dose of about 80 mg of fitusiran every month (or every four weeks). In yet other embodiments, the device may be used to prophylactically treat a patient with hemophilia A or B, with or without inhibitors, with a subcutaneous dose of about 20 mg of fitusiran every two months (or every eight weeks). In yet other embodiments, the device may be used to prophylactically treat a patient with hemophilia A or B, with or without inhibitors, with a subcutaneous dose of about 20 mg of fitusiran every month (or every four weeks). In yet other embodiments, the device may be used to prophylactically treat a patient with hemophilia A or B, with or without inhibitors, with a subcutaneous dose of about 10 mg of fitusiran every month (or every four weeks). In yet other embodiments, the device may be used to prophylactically treat a patient with hemophilia A or B, with or without inhibitors, with a subcutaneous dose of fitusiran at about 30 mg every month (or every four weeks). In yet other embodiments, the device may be used to prophylactically treat a patient with hemophilia A or B, with or without inhibitors, with a subcutaneous dose of fitusiran at about 5 mg every month (or every four weeks). In yet other embodiments, the device may be used to prophylactically treat a patient with hemophilia A or B, with or without inhibitors, with a subcutaneous dose of fitusiran at about 2.5 mg every month (or every four weeks). In yet other embodiments, the device may be used to prophylactically treat a patient with hemophilia A or B, with or without inhibitors, with a subcutaneous dose of fitusiran at about 1.25 mg every month (or every four weeks).

Accordingly, provided herein is a method of prophylactic treatment of a patient with hemophilia A or hemophilia B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of prophylactic treatment of a patient with hemophilia A or hemophilia B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Further provided herein is a method of reducing the frequency of bleeding episodes in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the frequency of bleeding episodes in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the ABR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 ml to about 1 ml delivery volumes (e.g., about 0.5 mL, about 0.6 mL, about 0.7 mL, about 0.8 mL, about 0.9 mL, or about 1 mL).

As an example, a method of reducing the ABR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the AjBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). The fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the AjBR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the AsBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 ml delivery volumes (e.g., about 0.5 mL, about 0.6 mL, about 0.7 mL, about 0.8 mL, about 0.9 mL, or about 1 mL).

As an example, a method of reducing the AsBR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

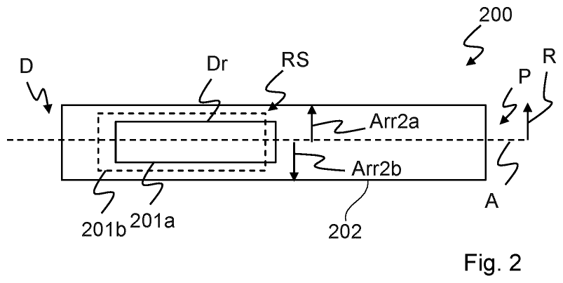

FIG. 2 illustrates a general second embodiment of a drug delivery device 200, e.g. of drug delivery device 100. Drug delivery device 200 may comprise:
A drug reservoir 201a, e.g. similar to medicament container 101a, and/or
A retaining/supporting member 201b that retains drug reservoir in position, and/or
A housing 202, e.g. similar to housing 102.

As indicated by two arrows Arr2a, Arr2b relative radial movement of drug reservoir 101a, 201a and/or needle 110 and housing 202 in a radial direction R may be possible due to at least one of the measures mentioned in this description, e.g.:
Usage of a soft material and/or of a flexible holding member, and/or
Usage of proximal supporting arms that may support drug reservoir 101a, 201a at a proximal end, e.g. on a flange of a syringe or on another proximal part.

Thus, the disclosure describes the concept, to decouple needle 110 and/or prefilled syringes (PFS) or other drug reservoirs 101a, 201a in an auto-injection pen or other drug delivery device 100, 200 from the housing 102, 202.

The disclosure may be used for auto-injection pens or for manually driven pens. From the user perspective, the use of device 100, 200 will be or may be identical to the usage of known drug delivery devices. The decoupling of needle 110 may allow needle 110 to some degree to free float perpendicular to the injection axis A.

Furthermore, the aim of the disclosure may be to reduce injection pain that may be caused to the patient my minor axial and/or radial movements that can occur during the injection. These movements could be the result from shaking hands or by intended or unintended movements. The disclosure appears especially relevant for patients that have difficulties holding the devise still, e.g. Parkinson disease, older patients, infants and small children that do self-administer the drug.

By decoupling needle 110 from housing 102, the use of device 100, 200, e.g. comprising a syringe 323, PFS may be much more comfortable for the user. Because there are fewer or smaller forces between the user skin and the needle 110. While the current development may be focused on autoinjectors and free-floating mechanism for the drugs Dr primary packaging, it might be applicable in any needle-based injection system, regardless of how it is actuated and who administers drug Dr.

FIGS. 3A to 3C illustrate a third embodiment of a drug delivery device 300, e.g. of drug delivery device 100, 200, etc. Drug delivery device 300 may comprise a main housing part 302 or main body, e.g. a cylindrical housing 302 or an essentially cylindrical housing 302. A piston rod or plunger 304 that may correspond to piston rod 104 may be arranged in the proximal part of housing 304. Piston rod 304 may have a tubular shape.

An inner housing 327 may be arranged within housing 302. Inner housing 327 may be shorter than main housing 302, e.g. it may have an axial length that is less than half of the axial length of housing 304. Inner housing 327 may have a proximal flange that extends radially inwards from a wall of inner housing 327, e.g. form an inner surface of the wall of inner housing 327.

A soft material 321 may be arranged within inner housing 327. Thus, soft material 321 may have no direct physical contact to housing 302. Soft material 321 may form a radial supporting member that is configured to support a drug reservoir 323 radially during injection of a drug Dr that may be comprised within drug reservoir 323. Drug reservoir 323 may be arranged within a retaining space RS.

Soft material 321 may have a Shore-A hardness below 90, 85, 80, 75, 70, 65, 60, 55 or below 50 and/or may comprise one of the materials mentioned in the first part of the description. Moreover, soft material 321 may have a tubular shape. An inner lumen of the tube may be surrounded by a wall of the tube. The inner lumen of the tube may form the retaining space RS for retaining the drug reservoir 101a, 323.

Drug reservoir 323 may be a prefilled syringe PFS. A resilient stopper (plug) 323a may be arranged movable within drug reservoir 323, PFS and may close drug reservoir 323 proximally. Plunger 304 may interact with stopper 323a in order to eject drug Dr that is comprised within drug reservoir 323 through a needle 310 that may correspond to needle 110. The optional blind hole may be arranged in a proximal facing face of stopper 323a.

Prefilled syringe PFS may comprise a distal conical portion for holding needle 310, a middle barrel portion, e.g. a cylindrical portion and a proximal flange Fl. Flange Fl may have a circular shape or a circular shape comprising two optional flat surfaces on opposite radial sides or side faces of flange Fl.

Drug delivery device 300 may comprise an axially movable needle cover (shield) 324, NC. Needle cover 324, NC may comprise:
A distal tubular portion, comprising a distal facing bearing surface BF adapted for contact with the skin S of a patient Pat, see also FIG. 3B,
Two proximally extending arms 324a, e.g. long arms of needle cover 324, NC that may extend from the distal tubular portion to a proximal end 324b of needle cover NC. The proximal end 324b may be arranged near a proximal end of the plunger/piston rod 304 in the inactivated state of plunger 304. Thus, needle cover NC may be used to trigger injection of drug Dr by unlocking plunger 304 or it may enable injection by unlocking plunger 304.
At least two arms 331, e.g. short arms 331 that are described below in more detail.

Drug delivery device 300 may comprise a rear sub assembly or rear part 325 that may be inserted in a proximal opening of housing 302. Rear sub assembly 325 may comprise:
An optional stem 325a that may be arranged within a hollow lumen of plunger 304, and
Two arms 328 that are described below in more detail.

A first low friction element 326a, e.g. a first ball bearing BB may be arranged on the distal ends of arms 328. The balls of ball bearing BB may abut on a proximally facing face of flange Fl. A second low friction element 326b, e.g. a second ball bearing BB may be arranged on a distally facing face of flange FI or on the flange of inner housing 327. In the first variant, the balls of the second ball bearing BB may abut the distally facing face of flange Fl. In the second variant, the balls of the second ball bearing BB may abut the flange of inner housing 327. Instead of inner housing 327 it is possible to use a flange that is similar to the flange of inner housing 327 but that is arranged on housing 302. However, inner housing 327 may ease assembly of drug delivery device 300, especially assembly of ball bearing(s) BB.

Instead of the first ball bearing BB and/or instead of the second ball bearing BB it may be possible to used plane surfaces, e.g. proximally and/or distally facing surfaces that are coated with a low friction material, e.g. PTFE or grease.

Thus, the at least one supporting arm 328 may carry a ball bearing BB on its distal end. The ball bearing may support the drug administration element, e.g. needle 310 axially, preferably via the drug reservoir 101a, 323, PFS.

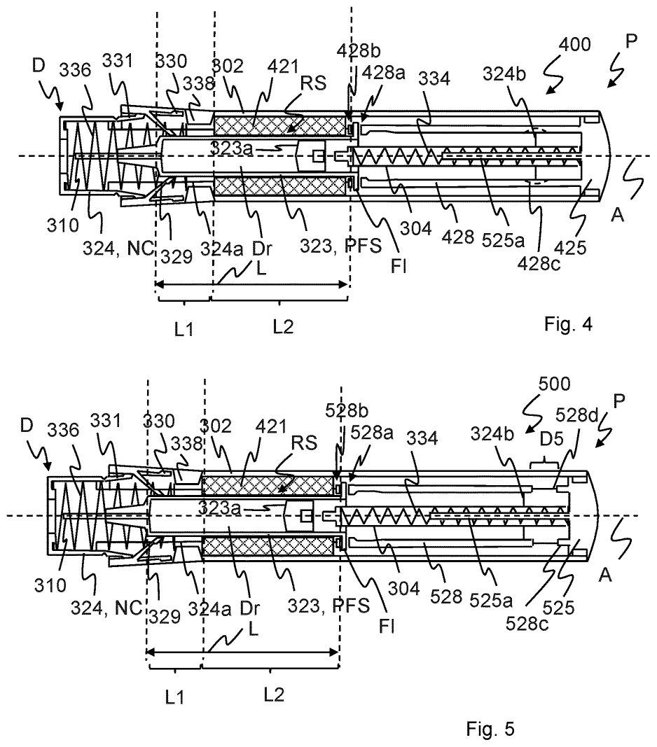

First arms 328 may form an axial supporting member, e.g. supporting arms that extend parallel to or along a plunger or piston rod 304 from rear part 325 in a distal direction. The axial supporting member, e.g. first arms 328 may be configured to support a drug administration element, e.g. needle 110, 310 axially. First arms 328 may be rigid arms or flexible arms. First arms 328 may comprise optional thinner portions, e.g. on its inner side and/or outer side, see thinner portions 428c in FIG. 4.

Second arms 329 may be arranged on a distal portion of housing 304. Second arms 329 may form a radial and/or axial supporting member, e.g. hinged arms, pre-injection supporting member. Second arms 329 may be rigid or flexible.

Thus, the least one supporting member mentioned above may comprise at least one pre-injection supporting member, e.g. two second arms 329. The at least one pre-injection supporting member, e.g. arms 329 may be configured to be in a first state before the drug administration element, e.g. needle 110, 310 is inserted into the tissue of a patient Pat. The at least one pre-injection supporting member 329 may be configured to be in a second state when the drug administration element, e.g. needle 110, 310 is inserted into the tissue of a patient Pat. In the first state-illustrated in FIG. 3A, the pre-injection supporting member, e.g. arms 329 may support the drug administration element, e.g. needle 310 radially and/or axially. The support may be indirectly via support of drug reservoir/container 323, PFS, e.g. on a distal part of the barrel portion of drug reservoir 323, PFS. In the second state-illustrated in FIG. 3B, the pre-injection supporting member does not support the drug administration element, e.g. needle 110, 310 or does support the drug administration element, needle 110, 310 less compared to the support in the first state.

The needle cover 324, NC may comprise pockets 330 on its inner side. Pockets 330 may be complementary to arms 329, e.g. at least on sides that are facing or that are directed to each other.

Needle cover 324, NC may comprise third arms 331. Third arms 331 may extend proximally from the tubular portion of needle cover 324, NC. Third arms 331 may be rigid arms or flexible arms. Third arms 331 may have the same angular position as second arms 329. Thus, second arms 329 may be moved by third arms 331 into pockets 330 when needle shield 324, NC is moved proximally during insertion of needle 310 into the skin S of patient Pat.

Drug delivery device 300 may comprise only two springs:
A drive spring 334 that may drive plunger or piston rod 302 during injection, and
A needle cover spring 336 that may bias needle cover 324, NC into the distal direction D.

Drive spring 334 that may be arranged within piston rod 304, e.g. on optional stem 325b. Alternatively, drive spring 304 may be arranged on the outside of piston rod 304. Drive spring 334 may abut against a proximal facing face at the bottom of the inner lumen of piston rod 304 and against a distal facing face of rear sub assembly/rear part 325.

Needle cover spring 336 may be arranged within distal tubular portion of needle cover 324, NC. Needle cover spring 336 may surround needle 310, conical part of drug reservoir 323 and/or a distal part of the barrel portion. Needle cover spring 336 may abut against an inner bottom surface of the tubular portion of needle cover 324, NC and against a protrusion 338 that is described in more detail in the following.

Housing 304 may comprise an inner bearing protrusion 338, e.g. a circular protrusion extending along the whole circumference or along parts of the circumference of an inner surface of housing 304. Bearing protrusion 338 may support soft material 321 and/or inner housing distally, e.g. during injection.

A distal length L1 of barrel portion of drug reservoir 323, PFS may not be covered by soft material 321. Contrary, a proximal length L2 may be covered by soft material 321. The sum of length L1 and L2 may be the overall length of drug reservoir 323, PFS, e.g. of a glass part of reservoir 323, PFS. Length L1 may be shorter than length L2. However length L2 may be at least one quarter of length L. Other possibilities for values of length L2 are mentioned in the first part of the description. The same ratios for length L1, L2 and length L may apply if length L corresponds to the overall length of the glass body of the syringe 323, PFS or to the overall length of another medicament container.

FIG. 3B illustrates the second state of drug delivery device 300, e.g. needle cover 324, NC is in its second position, see proximal end 324b that has moved proximally by a distance D3, see double headed arrow. Drug delivery device 300 is pressed against the skin S of a patient Pat thereby moving needle cover 324, NC proximally. The basic principle of operation of drug delivery device 300 may be the same as in known autoinjectors or other known drug delivery devices. Plunger 304 may be triggered by the axial displacement of needle cover 324, NC. A soft material 321 may surround syringe 323, PFS and may isolate it from the housing 302. Due to the low Shore-A hardness of soft material 321, wobbling of the syringe 323 in the radial R direction may be possible.

The positioning of syringe 323, PFS in an axial direction A may be realized through the flange FI being positioned between arms 328 of rear sub assembly 325 and inner housing 327. The contact surfaces may be on low friction element(s) 326a, 326b, e.g. at least one axial ball bearing BB or at least one PTFE coated surface. This may allow the PFS, 323 to radially move while being secured in its axial position.

Two arms 329, e.g. flexible arms may realize centering of needle 110, 310 prior to piercing skin S. During the injection phase needle cover 324, NC may disconnect arms 329 from syringe 323 and syringe 323, PFS may decouple from radial movement of the body or housing 302. During injection, flexible arms 329 that stabilize the PFS, 323, e.g. radially and/or axially may be pushed into pockets 330 in body/housing 302 by needle cover 324, NC. This may decouple syringe 323, PFS in radial R direction.

Due to the usage of arms 328, 329 and/or 331 and/or due to usage of soft material 321 as well as usage of low friction elements 326a, 326b drug reservoir 323, PFS may radially “float” during injection, e.g. a relative radial movement or displacement between housing 304 and drug reservoir 323, PFS carrying needle 110, 310 may be allowed, e.g. in order to reduce pain of patient P and/or to achieve other medical advantages and technical effects mentioned above. It may be of course be possible to use more or less measures or other measures to achieve radial “floating”. It may be possible to omit arms 329 and/or 331. Alternatively, it may be possible to omit soft material 321 and/or inner housing 327. Omitting arms 328 may also be an option.

FIG. 3C illustrates a cross section of device 300 on a section C-C illustrated in FIG. 3B. An example of a ball bearing BB is illustrated that may be used as low friction element 326a and/or 326b. Ball bearing BB may comprise:
Balls 340, e.g. at least three balls or at least eight balls,
A cage 342 that may hold balls 340, and
An optional housing part that may carry cage 342 and/or that may be used for mounting of ball bearing BB. Alternatively, cage 342 may have a housing function and/or a mounting function.

FIG. 4 illustrates a fourth embodiment of a drug delivery device 400, e.g. of drug delivery device 100. Drug delivery device 400 may comprise the same or similar parts as drug delivery device 100, 200 or 300, e.g. parts 302 (main housing part) to 324 (needle cover NC), 324a, 324b, etc., especially arms 329 and 331. A soft material 421 is modified compared to soft material 321. Soft material 421 is hold without a separate inner housing. Thus, soft material 421 may be adjacent to housing 302 (body) of drug delivery device 400, e.g. in direct physical contact. However, in another embodiment an inner housing may be used as well in drug delivery device 400. This inner housing may or may not have a proximal flange, as the design of arms 428 is different from the design of arms 328.

A rear sub assembly/rear part 425 may be similar to rear assembly 325. Thus, optional stem 325a may be present in rear assembly 425 too.

Two arms 428 may be used instead of arms 328. At least one supporting arm 328 or both arms 328 may comprise an aperture 428a on its distal end 428b. Aperture 428a may be configured to couple to flange Fl of drug reservoir 101a, 323, PFS, preferably to the flange Fl of a syringe PFS. A distal end portion 428b of arm 328 may be arranged distally of flange FI. Moreover, optional thinner portions 428c may be arranged on arm(s) 328, e.g. in order to raise flexibility of the arm(s) 328. Again, arms 328 may be rigid or may be flexible.

Thus, FIG. 4 illustrates another variant for the axial positioning of the syringe 323, PFS, e.g. via a flexible design of arms 428 of rear sub assembly 425. Flange Fl of syringe 323 may be clipped in arms 428 of rear sub assembly 425. Through flexible design of arms 428, a movement of distal ends 428b of arms 428 and/or of a proximal portion of syringe 323, PFS in radial R direction may be possible.

Again, decoupling of needle 310 may be reached by the usage of arms 329, 331, soft material 321 and/or usage of arms 428 and/or of thinner portions 428c. Decoupling of needle 310 may result in less pain of patient P and/or in other medical and/or technical effects. It is of course possible to use more or less measures or other measures to achieve radial “floating”. Thus, it may be possible to omit arms 329 and/or 331. Alternatively, it may be possible to omit soft material 321.

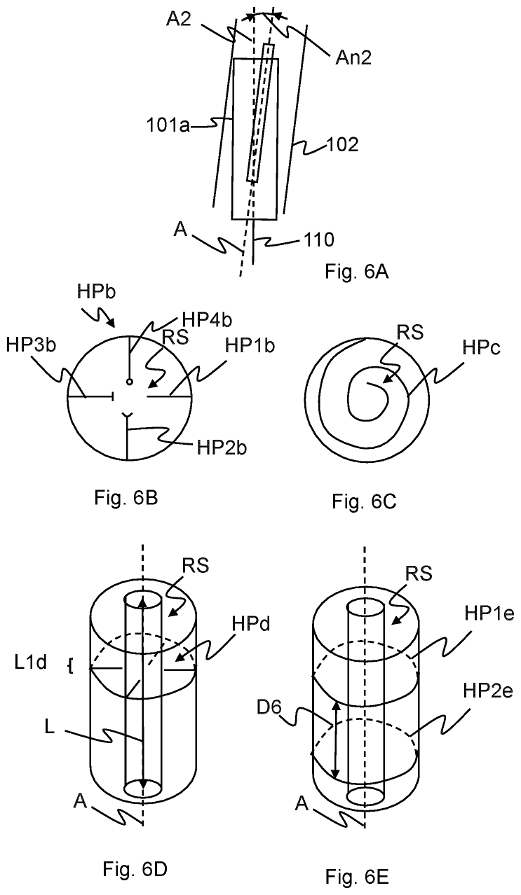

FIG. 5 illustrates a fifth embodiment of a drug delivery device 500, e.g. of drug delivery device 100. Drug delivery device 500 may comprise the same or similar parts as devices 100, 200, 300, 400, e.g. parts 302 (main housing part) to 324 (needle cover NC), 324a, 324b, etc., especially arms 329 and 331. Soft material 421 may be used in device 500 as well, e.g. with no inner housing. However, it is of course possible to use an inner housing also in device 500.

Rear sub assembly 525 may be similar to rear sub assembly/rear part 325. Thus, an optional stem 325a may also be present in device 500 in order to guide distal movement of piston rod 304 and/or of drive spring 334.

First arms 528 may be rigid or flexible. First arms 528 may have apertures 458a and distal end portions 528b. Thus, the distal ends of arms 528 may be similar to or same as distal ends of arms 428. However, arms 528 do not extend up to rear sub assembly 525 but only to coupling elements that are arranged between a respective arm 528 and rear sub assembly 525. Proximally of the coupling elements there may be optional second portions 528c. Alternatively, the proximal end of the coupling element(s) may be arranged directly on rear sub assembly 525.

Coupling element(s) may comprise at least one filament (rope) 528d. Alternatively, wires or other elements mentioned above may be used as coupling elements.

Thus, a proximal end of the at least one supporting arm 328 may be coupled to a proximal part of housing 102 or to a rear part 525 of the housing 102 that may be inserted into the housing 102 by at least one coupling element. The coupling element may be configured to transfer no axial pressure force or less axial pressure force compared to axial tension force transferable by the coupling element. As mentioned already, the at least one coupling element may comprise or may consist of at least one filament.

The coupling element may bridge an axial distance D5, e.g. a distance within the range of 5 mm to 30 mm or within the range of 10 mm to 20 mm.

Again, decoupling of needle 310 may be reached by the usage of arms 329, 331, soft material 321 and/or usage of arms 528 and/or of thinner portions similar to thinner portions 428c (see FIG. 4) and/or coupling element, e.g. filament 528d. Decoupling may result in less pain of patient P and/or in other medical and/or technical effects, e.g. as mentioned above. It is of course possible to use more or less measures or other measures to achieve radial “floating”. Thus, it may be possible to omit arms 329 and/or 331. Alternatively, it may be possible to omit soft material 321.

Thus, another variant for the axial positioning of the PFS, 323 may be to connect the element holding flange FI to rear sub assembly 525 via a rope or wire 528d. The rope or wire 528d may only transfer tension, e.g. may hold syringe 323, PFS when plunger 324 is released. Further, the rope or other coupling element may decouple the syringe 323, PFS radially and in terms of pressure. The pressure on syringe 323, PFS during insertion of needle 110 in this case may be hold by flexible arms 329 that are meant for positioning the syringe 323, PFS before the injection starts and/or by soft material 421. Additionally or alternatively, plunger 304 may provide axial support during insertion of needle 110, 310.

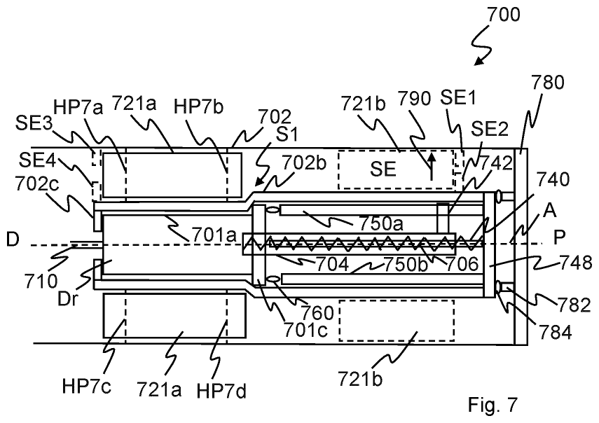

FIGS. 6A to 6E illustrate variants of further embodiments. As is illustrated in FIG. 6A, decoupling may allow pivoting of the housing 102, 202, etc. relative to an axis A2 of drug reservoir 101a, 202a, etc. or vice versa, preferably during usage of drug delivery device 100 to 500, e.g. during injection. Thus, there may be an angle An2 between axis A2 and longitudinal axis A of drug delivery device 100 to 500 having a value within the range of 1 degree to 5 degree or within another appropriate range.

FIG. 6B illustrates holding portion HPb that may be used instead of soft material 321 in drug delivery device 100 to 500. It may be possible to use at least three holding portion or at least four holding portions HP1b to HP4b or more than four holding portions. The holding portions may all have the same design or may have different designs from each other. The holding portions may extend only radially inwards or radially and axially from housing 102, etc. to retaining space RS or to drug reservoir 101a, 323, etc. Thus, the holding portions may be radially extending arms. FIG. 6B illustrates four different designs:
Holding portion HP1b may comprise no special free end,
Holding portion HP2b may comprise an arc shaped free end,
Holding portion HP3b may comprise a straight free and that is arranged perpendicular to the main part and/or axis of holding portion HP3b, and
Holding portion HP4b may comprise a soft or hard end portion, e.g. in ball shape or in the shape of a sphere or of a semi-sphere.

The free ends may be configured or may be in physical contact with the drug reservoir 101a, 323, PFS. Other designs are possible as well. The free ends of holding portions HP1b to HP4b may prevent damage of breakable drug reservoirs 101a, 323, PFS.

Thus, the radial supporting member may comprise at least one flexible holding portion HP. The at least one flexible holding portion HP may comprise at least one flexible element. The amount of flexibility of the flexible element may be chosen such that bending of the at least one flexible element is possible by radial forces during usage of the drug delivery device 100 to 500 or of another device. The flexible holding portion may comprise at least one straight element extending radially inwards up to a retaining space RS for the drug reservoir 101b, 323, etc.

As is illustrated in FIG. 6C a spirally shaped holding portion HPc may be used. The flexible holding portion HP may comprise a spirally wound element. An axis around which a spiral of the spirally wound portion is wound may be parallel to the longitudinal axis A or may corresponds to the longitudinal axis A of the drug delivery device 100 to 500 or of another device. Drug reservoir 323, PFS etc. may be arranged within a retaining space RS that may also be arranged within the center of the spiral. The spiral may be formed by an elongated arm or by a sheet material as mentioned above.

As is illustrated in FIG. 6D, only one holding portion HPd may be used to hold the drug reservoir, e.g. holding portion HPb or HPc. Holding portion HPd may have a length L1d that is much shorter than the length of the drug reservoir, e.g. less than 30 percent or less than 10 percent of length(s) L mentioned above. Holding portion HPd may be arranged on a proximal part of drug reservoir, e.g. in order to allow pivoting relative to housing 102, etc. Alternatively only a short tubular or a ring shaped soft material may be used as holding portion HPd.

As is illustrated in FIG. 6E, at least two holding portions HP1e and HP2e may be used in drug delivery device 100 to 500 or in another drug delivery device, see e.g. HPb, HPc, or short ring shaped soft material portions. Adjacent holding portion HP1e, HP2e, etc. may have an axial distance D6 that may be in the range of 5 mm to 30 mm or within another appropriate range.

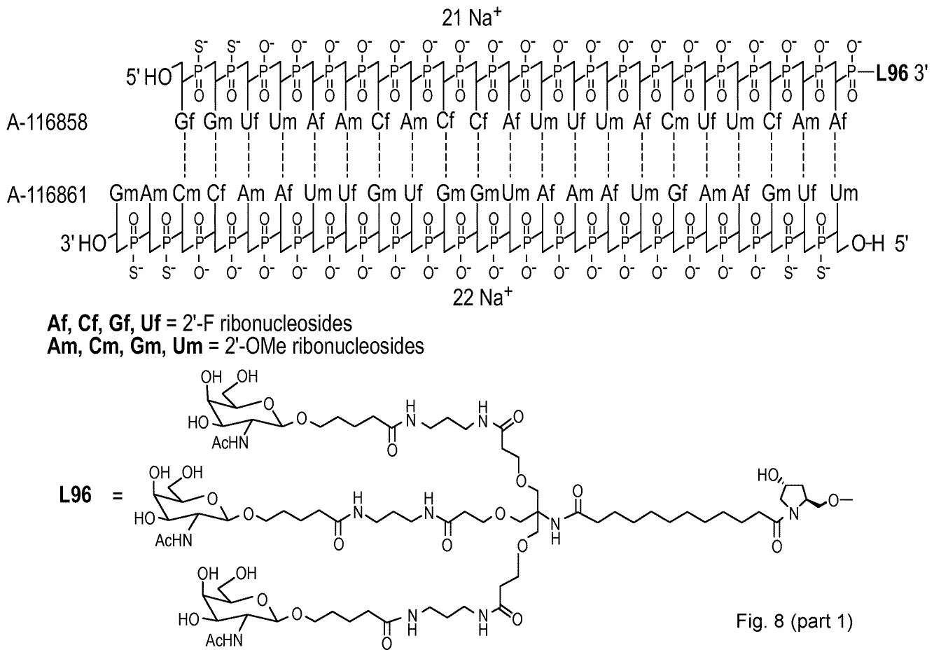

FIG. 7 illustrate a last embodiment, i.e. a drug delivery device 700. Drug delivery device 700 may comprise:
A medicament container 701a, e.g. a medicament container comprising a flange 701c,
A main housing part 702 that forms an outer housing, e.g. comprising an essentially cylindrical shape or a cylindrical shape,
An inner housing part 702b comprising an optional distal stop element 702c,
A piston rod 704,
A driving mechanism 706, e.g. a drive spring,
An actuating element that is not illustrated, e.g. an axially movable needle sleeve (shroud), and
An optional needle 710.

Again a drug Dr may be comprised within medicament container 701a. Drug Dr may be one of the drugs as mentioned in the drug list above.

There may be a soft material portion 721a and/or a soft material portion 721b arranged between outer housing 702 and inner housing 702b. Alternatively, only one longer soft material portion may be used instead of both soft material portions 721a and 721b.

In another embodiment, flexible holding portion HP7a to HP7d, e.g. arms or beams or a sheet of material, may be used instead of soft material portion 721a or in addition to soft material portion 721a. Further holding portions may be arranged in other cross sections, e.g. in a cross section of drug delivery device 700 that is perpendicular to the cross section that is illustrated in FIG. 7 and that may extend also through the longitudinal axis A of drug delivery device 700. Moreover, flexible holding portions, e.g. arms or beams may be used in the proximal half, e.g. between the outer housing 702 and the wider portion of inner housing 702b.

Inner housing part 702b may comprise two cylindrical portions. Inner housing 702b may be a part integrally formed, e.g. using injection molding. In another embodiment, inner housing may comprise not only one part but at least two parts which may be assembled together, e.g. using a screw thread or a snap connection or another connection.

A distal part of inner housing 702b may have a smaller diameter compared to the proximal part of inner housing 702b. The inner diameter of the distal part of housing 702b may be adapted to the outer diameter of a main part of medicament container 701a.

The inner diameter of the proximal part of housing 702b may be adapted to the outer diameter of flange 701c, e.g. to the largest outer diameter if flange 701c comprises at least one flat, typical two flats on opposite sides.

In another embodiment, inner housing 702b may have a constant diameter or an approximately constant diameter along the main part of its length, i.e. along at least 90 percent of its axial length. Thus, it may be possible to insert a syringe comprising a flange 701c laterally or to use a medicament container that does not have a flange.

Piston rod 704, e.g. a plunger, may have a cylindrical shape that is hollow. Thus, drive spring 706 may be arranged within piston rod 704. An optional pin 740 may be used to stabilize drive spring 706 within piston rod 704. In another embodiment, drive spring 706 may be arranged at the outside of piston rod 704.

Moreover, piston rod 704 may comprise at least one wing or protrusion 742 that extends radially. Protrusion 742 may be used to release piston rod 704 in order to initiate injection of drug Dr.

The axial movable needle sleeve or shroud may comprise to long arms extending proximally P. Proximal ends of these long arms may be configured to interact with at least one lateral protrusion of piston rod 704. The long arms may extend within inner housing 702b or outside of inner housing 702b but inside of outer housing 702. The length of the lateral protrusions of piston rod 704 may be adapted accordingly.

A rear part 748 of inner housing 702b may hold two rigid arm 750a, 750b which may extend distally from rear part 748. Rear part 748 may be constructed as a lid that is adapted to close inner housing 702b proximally. However, complete closure is optional.

Each rigid arm 750a, 750b may carry a respective resilient element 760, e.g. a spring element, especially a plastic spring element. Resilient elements 760 may bias flange 701c or another proximal part of a medicament container 701a axially, e.g. distally. There may be radial degrees of freedom between resilient element(s) 760 and flange 701c or another proximal part. However, this radial degree of freedom may be not necessary within inner housing 702b since inner housing 702b is radially movable with regard to outer housing 702. Axial movement between inner housing 702b and outer housing may not be possible as is described below in more detail.

A rear part 780 of outer housing 702 may be shaped as a lid that may close outer housing 702 proximally. However, complete closure is optional. There, may be at least one optional arm 782 or at least one optional other supporting structure that may extend distally from lid or rear part 780 of outer housing 702. An axial supporting element 784, e.g. ball bearing as mentioned above or radially extending surface, e.g. surface coated with Teflon (may be a trade mark) or other material of low friction coefficient may be arranged on the at least one optional arm 782 or on the at least one optional other supporting structure on rear part 780. Axial supporting element 784 may prevent proximal movement of inner housing 702b relative to outer housing 702. However, radial movement of inner housing, especially of the proximal end of inner housing 702b relative to outer housing 702 may be possible, see arrow 790.

This radial movement degree of freedom may be greater compared to other embodiments described above. Moreover, piston rod 104 may be always parallel to medicament container 701a allowing full injection of drug Dr within a short time, e.g. contact between piston rod 704 and medicament container 701a, especially a glass part of medicament container 701a may be avoided.

There may be a step S1 at the border of a narrower distal part of inner housing 702c and a wider proximal part of 702c. Step S1 may be used to prevent distal movement of inner housing 702c, e.g. by abutment between step S1 and soft material 721a or a rigid holder for soft material 721a. However, other stop elements SE1 to SE4 may be used to prevent distal movement of inner housing 702b. Stop elements SE1 and/or SE3 may be arranged on outer housing 702. Stop elements SE2 and/or SE4 may be arranged on inner housing 702c. Combinations of stop elements SE1 to SE4 may be used, e.g. as long as there is a radial space between opposite stop elements on inner housing 702b and on outer housing 702 allowing radial displacement of inner housing 702b relative to outer housing 702. Other axial fixing elements may be used in other embodiments.

Assembling may be performed in the following way, e.g. for the embodiment illustrated in FIG. 7:
Inner housing 702b is assembled by introducing medicament container 701a (syringe/cartridge), drive mechanism 706 and plunger 704 together with rear part 748,
Soft material portions 721a and/or 721b and/or holding portions HP7a to HP7b may be arranged on the outside of inner housing 702b prior or after assembling of inner housing 702b. In an embodiment, soft material portions 721a and/or 721b and/or holding portions HP7a to HP7b may be integral parts of inner housing 702b,
Soft material portions 721a and/or 721b and/or holding portions HP7a to HP7b as well as assembled inner housing may be inserted into outer housing 702, and
Outer housing 702 is closed by rear part 780.

An optional priming step may be performed during or after assembling.

Spoken with other words, drug delivery device 100 to 500 or another drug delivery device may comprise:
A housing 102, etc. for receiving a drug reservoir 101a,
wherein a drug administration element 110, 310 may be fluidically coupled or coupleable to drug reservoir 101a, drug administration element 110, 310 defining a drug outlet of drug delivery device 100 to 500, and/or
At least one supporting member that may be mechanically coupled or may be coupleable to the drug administration element 110, 310 in order to support drug administration element 110, 310 relative to housing 102, 302,
wherein the at least one supporting member may be configured to allow a relative radial displacement of the housing 102, 302, etc. relative to the drug administration element 110, 310 during a drug delivery operation.

A method for decoupling a drug administration element, e.g. needle 110, 310, etc. and at least one part of a drug delivery device 100 to 500 or another drug delivery device may comprise:
Using a radial supporting member 101b comprising at least one of a soft material for holding a drug reservoir 101a or a flexible holding portion HP for holding the drug reservoir 101a, 323, PFS,
wherein at least one of the soft material 321 or flexible holding portion HP may be configured to allow a relative radial R displacement of at least a portion of drug reservoir 101a, 323, PFS relative to a housing 102, 302 of drug delivery device 100 to 500 during insertion of the drug administration element arranged or arrangeable on the distal end D of drug reservoir 101a, etc. into the tissue of a patient Pat, e.g. into skin S.

Although embodiments of the present disclosure and their advantages have been described in detail, it should be understood that various changes, substitutions and alterations can be made therein without departing from the spirit and scope of the disclosure as defined by the appended claims. For example, it will be readily understood by those skilled in the art that many of the features, functions, processes and methods described herein may be varied while remaining within the scope of the present disclosure. Moreover, the scope of the present application is not intended to be limited to the particular embodiments of the system, process, manufacture, method or steps described in the present disclosure. As one of ordinary skill in the art will readily appreciate from the disclosure of the present disclosure, systems, processes, manufacture, methods or steps presently existing or to be developed later that perform substantially the same function or achieve substantially the same result as the corresponding embodiments described herein may be utilized according to the present disclosure. Accordingly, the appended claims are intended to include within their scope such systems, processes, methods or steps. Further, it is possible to combine embodiments mentioned in the first part of the description with examples of the second part of the description which relates to FIGS. 1 to 8.

1-16. (canceled)

17. A drug delivery device, comprising:
a housing for receiving a drug reservoir,
a drug administration element fluidically coupled to the drug reservoir, the drug administration element defining a drug outlet of the drug delivery device, and
at least one supporting member coupled to the drug administration element for supporting the drug administration element relative to the housing,
wherein the at least one supporting member is configured to allow a radial displacement of the housing relative to the drug administration element during a drug delivery operation comprising injection of a drug contained in the drug reservoir.

18. The drug delivery device according to claim 17, wherein the at least one supporting member comprises a radial supporting member configured to support the drug reservoir radially during the drug delivery operation.

19. The delivery device according to claim 18, wherein the radial supporting member at least partially comprises a soft material having a Shore-A hardness value below 90.

20. The delivery device according to claim 19, wherein the radial supporting member at least partially comprises a soft material having a Shore-A hardness value below 80.

21. The delivery device according to claim 20, wherein the radial supporting member at least partially comprises a soft material having a Shore-A hardness value below 70.

22. The delivery device according to claim 21, wherein the radial supporting member at least partially comprises a soft material having a Shore-A hardness value below 60.

23. The delivery device according to claim 22, wherein the radial supporting member at least partially comprises a soft material having a Shore-A hardness value below 50.

24. The drug delivery device according to claim 19, wherein the soft material comprises a tubular shape, wherein an inner lumen of the tube is surrounded by a wall of the tube and wherein the inner lumen of the tube forms a retaining space for retaining the drug reservoir.

25. The drug delivery device according to claim 18, wherein the radial supporting member comprises at least one flexible holding portion comprising at least one flexible element,
wherein the degree of flexibility of the flexible element is chosen such that bending of the at least one flexible element is possible by radial forces during the drug delivery operation.

26. The drug delivery device according to claim 25,
wherein the flexible holding portion comprises at least one straight element extending radially inwards up to a retaining space for the drug reservoir, or
wherein the flexible holding portion comprises a spirally wound element.

27. The drug delivery device according to claim 17, wherein the at least one supporting member comprises at least one pre-injection supporting member,
wherein the at least one pre-injection supporting member is configured to be in a first state before the drug administration element is inserted into a tissue of a patient, and
wherein the at least one pre-injection supporting member is configured to be in a second state when the drug administration element is inserted into the tissue of a patient,
wherein in the first state, the pre-injection supporting member supports the drug administration element, and
wherein in the second state, the pre-injection supporting member does not support the drug administration element or does support the drug administration element less compared to the support in the first state.

28. The drug delivery device according to claim 17, wherein the at least one supporting member comprises an axial supporting member that is configured to support the drug administration element axially, and
wherein the axial supporting member comprises at least one supporting arm that extends in a distal direction.

29. The drug delivery device according to claim 28, wherein the at least one supporting arm carries a low friction material that enables radial movement of the drug reservoir relative to the at least one supporting arm with lower friction compared to a main material of the at least one supporting arm,
wherein the low friction material comprises or consists of PTFE, and/or
wherein the at least one supporting arm carries a ball bearing on its distal end, wherein the ball bearing supports the drug administration element axially via the drug reservoir.

30. The drug delivery device according to claim 28, wherein the at least one supporting arm comprises an aperture on its distal end configured to couple to a flange of the drug reservoir, and/or wherein a proximal end of the at least one supporting arm is coupled to a proximal part of the housing or to a rear part of the housing by at least one coupling element, wherein the coupling element is configured to transfer no axial pressure force or less axial pressure force compared to axial tension force transferable by the coupling element, wherein the at least one coupling element comprises at least one filament.

31. The drug delivery device according to claim 17, wherein a retaining space for the drug reservoir and/or the drug reservoir is radially surrounded by a tubular distance space that does not comprise a radial supporting element or that does only comprise at least one pre-injection supporting element removed from the drug reservoir before or during injection,
wherein the tubular distance space extends along a length that is at least 90 percent of the axial length of the drug reservoir, and
wherein the drug reservoir is not supported radially or is only radially supported at its proximal end during the drug delivery operation.

32. The drug delivery device according to claim 17, wherein the housing is an outer housing of the drug delivery device,
wherein the drug delivery device comprises an inner housing that is arranged within the outer housing, and
wherein the at least one supporting member is configured to allow radial displacement of the inner housing relative to the outer housing.

33. Drug delivery device according to claim 32, wherein the drug delivery device comprises an axial support element that is arranged on the outer housing, and
wherein the axial support element is configured to allow radial displacement of a proximal end of the inner housing relative to a proximal end of the outer housing.

34. Drug delivery device according to claim 17, wherein the drug delivery device comprises at least one of:
a drive spring that is configured to drive a plunger, or
an axially movable needle cover that is axially movable along a longitudinal axis of the drug delivery device form a first position to a second position that is proximal with regard to the first position, wherein the needle cover is configured to cover a needle arranged on the drug reservoir in the first position and wherein the needle cover is configured to expose the needle when it is in the second position.

35. A method for decoupling a drug administration element and at least one part of a drug delivery device, the drug delivery comprising:
a housing for receiving a drug reservoir,
a drug administration element fluidically coupled to the drug reservoir, the drug administration element defining a drug outlet of the drug delivery device, and
at least one supporting member coupled to the drug administration element for supporting the drug administration element relative to the housing,
wherein the at least one supporting member is configured to allow a radial displacement of the housing relative to the drug administration element during a drug delivery operation comprising injection of a drug contained in the drug reservoir, the method comprising
using the radial supporting member comprising at least one of at least one soft material for holding a drug reservoir or at least one flexible holding portion for holding the drug reservoir,
wherein the at least one of the at least one soft material or the at least one flexible holding portion is configured to allow a relative radial displacement of at least a portion of the drug reservoir relative to a housing of the drug delivery device during insertion of a drug administration element arranged or arrangeable on the distal end of the drug reservoir into the tissue of a patient.

36. A drug delivery device comprising:
a housing for receiving a drug reservoir,
a drug administration element is fluidically coupled to the drug reservoir, the drug administration element defining a drug outlet of the drug delivery device, and
at least one supporting member coupled to the drug administration element for supporting the drug administration element relative to the housing,
wherein the at least one supporting member is configured to allow a radial displacement of the housing relative to the drug administration element during a drug delivery operation,
wherein the at least one supporting member comprises a radial supporting member that is configured to support the drug reservoir radially during injection of a drug that is contained in the drug reservoir,
wherein the radial supporting member comprises a soft material having a Shore A hardness value below 90, and wherein the soft material extends from an inner wall of the housing to the drug reservoir.
